(19) United States
(12) Patent Application Publication
(10) Pub. No.: US 2022/0273714 A1
(43) Pub. Date: Sep. 1, 2022

(54) T-CELL RECEPTORS AND METHODS OF USE THEREOF

(71) Applicant: THE REGENTS OF THE UNIVERSITY OF CALIFORNIA, Oakland, CA (US)

(72) Inventors: Hideho OKADA, Oakland, CA (US); Diego A. CARRERA, Oakland, CA (US); Hirokazu OGINO, Oakland, CA (US)

(21) Appl. No.: 17/628,383

(22) PCT Filed: Jul. 17, 2020
(86) PCT No.: PCT/US2020/042611

§ 371 Date: Jan. 19, 2022

Related U.S. Application Data
(60) Provisional application No. 62/876,561, filed on Jul. 19, 2019.

(51) Int. Cl. A61K 35/17; A61P 35/00; C07K 14/725

(52) U.S. Cl. CPC A61K 35/17; A61P 35/00; C07K 14/7051

(57) ABSTRACT

The present disclosure relates to T-cell receptors (TCRs) and related antigen-binding constructs that selectively target a tumor-specific isoform of human RAD54 Homolog B (RAD 54B). Further disclosed are the antigen-binding constructs specific for binding the peptide in a peptide/MHC complex, as well as the sequences of complementary determining regions of the TCRs.

### CROSS-REFERENCE TO RELATED PATENT APPLICATIONS

The present patent application claims benefit of priority to U.S. Provisional Patent Application No. 62/876,561, filed Jul. 19, 2019. The disclosure of the above-referenced application is herein expressly incorporated by reference in its entirety, including any drawings.

### STATEMENT AS TO RIGHTS TO INVENTIONS MADE UNDER FEDERALLY SPONSORED RESEARCH AND DEVELOPMENT

This invention was made with government support under grants nos. R21 NS093654 and R35 NS105068 awarded by The National Institutes of Health. The government has certain rights in the invention.

### FIELD

The present disclosure relates to T-cell receptors (TCRs) and related antigen-binding constructs that selectively target a tumor-specific isoform of human RAD54 Homolog B (RAD54B).

### INCORPORATION OF THE SEQUENCE LISTING

The content of the electronically submitted sequence listing (Name: 048536_648001WO_Sequence_Listing_ST25.txt, Size: 23,327 bytes; and Date of Creation: Jul. 17, 2020) is herein incorporated by reference in its entirety.

### BACKGROUND

Gliomas are the most common primary brain tumors. The current mainstay of treatment includes surgery followed by a combination of chemotherapy and radiation therapy. Regardless of their stage at diagnosis, gliomas are considered to be malignant due to their invasive growth, resistance to therapy, and recurrence, which ultimately leads to patient death. Within the field of brain tumor immunotherapy, the paucity of targetable molecules make the treatment of gliomas very challenging, as not all cells within a tumor can be targeted in the same way (intratumoral heterogeneity) and not all gliomas express the same targets (interpatient heterogeneity). Immunotherapeutic modalities targeting non-mutated proteins that are specific to glioma may offer safe and effective treatment options for patients. The tumor-specific isoform of RAD54B, a DNA repair and recombination protein, is expressed at high levels in a vast majority of malignant gliomas, and the expression is uniform within individual tumors (not heterogeneous), thereby making this an attractive immunotherapy target. Immatics Biotechnologies GmbH has developed GAPVAC-Peptide Warehouse, a library of HLA-class I non-mutated peptides derived from primary WHO Grade IV glioma samples (Hilf, N., et al. (2019). Nature, 565(7738), 240), including a peptide derived from a cancer-specific isoform of RAD54B, to be used in a peptide vaccine.

Potential challenges with peptide vaccine-based therapies include poor immunogenicity in the absence of an adjuvant and susceptibility to enzymatic degradation, and thus improved compositions and methods for targeting cancer cells expressing cancer-specific isoforms of RAD54B are needed.

### SUMMARY

This section provides a general summary of the disclosure, and is not comprehensive of its full scope or all of its feature.

In one aspect, provided herein is an antigen-binding construct comprising i) a TCRα variable region comprising a complementary determining region (CDR) 3 having the amino acid sequence of SEQ ID NO: 8 or a variant thereof having at least 80% sequence identity to the amino acid sequence of SEQ ID NO: 8; and ii) a TCRβ variable region comprising a CDR3 having the amino acid sequence of SEQ ID NO: 11 or a variant thereof having at least 80% sequence identity to the amino acid sequence of SEQ ID NO: 11, wherein the antigen-binding construct is specific for the peptide SLYKGLLSV (SEQ ID NO: 1) in a peptide/MHC complex.

In some embodiments, according to any of the antigen-binding constructs described above, iii) the TCRα variable region further comprises a CDR1 from the TCRα variable region of SEQ ID NO: 12 or a variant thereof having at least 80% sequence identity to the CDR1 from the TCRα variable region of SEQ ID NO: 12; iv) the TCRα variable region further comprises a CDR2 from the TCRα variable region of SEQ ID NO: 12 or a variant thereof having at least 80% sequence identity to the CDR2 from the TCRα variable region of SEQ ID NO: 12; v) the TCRβ variable region further comprises a CDR1 from the TCRβ variable region of SEQ ID NO: 14 or a variant thereof having at least 80% sequence identity to the CDR1 from the TCRβ variable region of SEQ ID NO: 14; and/or vi) the TCRβ variable region further comprises a CDR2 from the TCRβ variable region of SEQ ID NO: 14 or a variant thereof having at least 80% sequence identity to the CDR2 from the TCRβ variable region of SEQ ID NO: 14.

In some embodiments, according to any of the antigen-binding constructs described above, the TCRα variable region comprises the amino acid sequence of SEQ ID NO: 12 or a variant thereof having at least 80% sequence identity to the amino acid sequence of SEQ ID NO: 12 and/or the TCRβ variable region comprises the amino acid sequence of SEQ ID NO: 14 or a variant thereof having at least 80% sequence identity to the amino acid sequence of SEQ ID NO: 14.

In some embodiments, according to any of the antigen-binding constructs described above, the construct further comprises a TCRα constant region and/or a TCRβ constant region. In some embodiments, the TCRα constant region comprises the amino acid sequence of SEQ ID NO: 13 or a variant thereof having at least 80% sequence identity to the amino acid sequence of SEQ ID NO: 13 and/or the TCRβ constant region comprises the amino acid sequence of SEQ ID NO: 15 or a variant thereof having at least 80% sequence identity to the amino acid sequence of SEQ ID NO: 15. In other embodiments, the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

In some embodiments, according to any of the antigen-binding constructs described above, the construct is a multimer comprising i) a first polypeptide comprising the TCRα variable region, and ii) a second polypeptide comprising the TCRβ variable region. In some embodiments, the first polypeptide comprises the amino acid sequence of SEQ ID NO: 2 or a variant thereof having at least 80% sequence identity to the amino acid sequence of SEQ ID NO: 2 and/or the second polypeptide comprises the amino acid sequence of SEQ ID NO: 3 or a variant thereof having at least 80% sequence identity to the amino acid sequence of SEQ ID NO: 3. In other embodiments, the first polypeptide comprises the amino acid sequence of SEQ ID NO: 16 or a variant thereof having at least 80% sequence identity to the amino acid sequence of SEQ ID NO: 16 and/or the second polypeptide comprises the amino acid sequence of SEQ ID NO: 17 or a variant thereof having at least 80% sequence identity to the amino acid sequence of SEQ ID NO: 17.

In some embodiments, according to any of the antigen-binding constructs described above, the construct is a T-cell receptor or an antigen-binding derivative or fragment thereof.

In some embodiments, according to any of the antigen-binding constructs described above, the MHC molecule in the peptide/MHC complex is HLA-A*02:01.

In another aspect, provided herein is nucleic acid encoding an antigen-binding construct according to any of the embodiments described above.

In another aspect, provided herein is a host cell comprising nucleic acid according to any of the embodiments described above, wherein the antigen-binding construct is capable of being expressed in the host cell. In some embodiments, the nucleic acid encoding the antigen-binding construct is heterologous to the host cell. In some embodiments, the host cell is a T-cell. In some embodiments, the T-cell is a CD8+ T-cell.

In another aspect, provided herein is a method of preparing a T-cell comprising or capable of expressing an antigen-binding construct according to any of the embodiments described above, comprising introducing nucleic acid encoding the antigen-binding construct into an input T-cell, wherein the antigen-binding construct is capable of being expressed in the input T-cell following introduction of the nucleic acid.

In another aspect, provided herein is a method of inducing an immune response to an isoform of RAD54B comprising the peptide SLYKGLLSV (SEQ ID NO: 1) in a subject, comprising administering to the subject a T-cell comprising or capable of expressing an antigen-binding construct according to any of the embodiments described above.

In another aspect, provided herein is a method of treating a disease or condition characterized by an isoform of RAD54B comprising the peptide SLYKGLLSV (SEQ ID NO: 1) in a subject in need thereof, comprising administering to the subject a T-cell comprising or capable of expressing an antigen-binding construct according to any of the embodiments described above.

In some embodiments, according to any of the methods of inducing an immune response or treating a disease or condition described above, the T-cell is autologous to the subject.

In some embodiments, according to any of the methods of inducing an immune response or treating a disease or condition described above, the subject has or is at risk of developing a cancer characterized by expression of a RAD54B isoform comprising RAD54B₆₁₈₋₆₂₆. In some embodiments, the cancer is a glioma. In some embodiments, the glioma is an astrocytoma, an oligodendroglioma, or a glioblastoma.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 shows a gene model for exon usage of the RAD54B protein by various isoforms.

FIG. 2 shows exon 11 of RAD54B mRNA expression in normal peripheral organs using GTEx database.

FIG. 3A shows expression of RAD54B as determined by single-cell RNAseq, arranged by by quartile of expression level, showing that RAD54B is expressed in less than 20% of sequenced cells of indicated cell lineages composing the normal brain. OPC: Oligodentrocyte progenitor cell; ODC: Oligodendrocyte. FIG. 3B shows the expression of RAD54B as determined by bulk RNAseq of human brain development, showing its expression in the adult brain is minimal (below 0.995 Log 2 RPKM).

FIG. 4A shows results for immunohistochemistry assays for the expression of RAD54B₆₁₈₋₆₂₆ in normal brain tissue, primary glioma, and recurrent glioma. FIG. 4B shows the t-test results.

FIG. 4C shows the quantification of RAD54B₆₁₈₋₆₂₆ mRNA expression by quantitative PCR in normal brain tissue, primary glioma, and recurrent glioma, measured by ratio to GAPDH expression.

FIG. 5A shows results for RAD54B₆₁₈₋₆₂₆-specific tetramer/dextramer staining of T-cells stimulated with RAD54B₆₁₈₋₆₂₆. FIG. 5B shows results for IFNγ ELISA of CD8+/tetramer+/dextramer+ T-cells stimulated with RAD54B₆₁₈₋₆₂₆, a negative control peptide, or no peptide. Stimulation with OKT3 antibody was included as a positive control.

FIG. 6 shows the splicing pattern for RAD54B in normal tissue and the splicing patterns for 3 different isoforms of RAD54B isolated from glioma. Exon 11 according to GTEx nomenclature is indicated in the box.

FIG. 7 shows the exemplary expression level of three different RAD54B isoforms in GBM.

### DETAILED DESCRIPTION OF THE DISCLOSURE

Provided herein are T-cell receptors (TCRs) and related antigen-binding constructs that target the peptide SLYKGLLSV (SEQ ID NO: 1) in a peptide/MHC complex. It is shown here that this peptide is expressed in certain cancer cells, including gliomas, but not expressed or expressed at very low levels in normal cells. This occurs as a result of the peptide being encoded by exon 11 of the RAD54B gene, which is retained in some cancer cells but generally not in normal cells. Further, it has been shown that the peptide (RAD54B₆₁₈₋₆₂₆) presentation on HLA-A*02:01 was detected only in cancer specimens but not in normal tissues. Moreover, it is shown that expression of this peptide was upregulated with tumor recurrence. It has also been suggested that this peptide presentation is up-regulated by irradiation in certain GBM cell lines. In addition, the present disclosure provides T-cells that can be selectively stimulated to secrete IFNγ when incubated with the peptide SLYKGLLSV (SEQ ID NO: 1), demonstrating the antigen specificity of the TCRs expressed by these T-cells. The data suggests that this epitope can be a target of CAR-T therapy. These results suggest the suitability of the TCRs and related constructs described herein for use in therapeutic methods of treating cancers characterized by expression of this peptide, such as adoptive T-cell therapy with T-cells engineered to express the TCR or related antigen-binding construct. Accordingly, also provided herein are methods of treating a cancer characterized by expression of a cancer-specific isoform of RAD54B including the peptide SLYKGLLSV (SEQ ID NO: 1) employing a TCR or related antigen-binding construct described herein.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

### Definitions

The terms “polynucleotide” and “nucleic acid” are used interchangeably herein and refer to a polymeric form of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases.

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

“Binding” as used herein refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵M. “Affinity” refers to the strength of binding, and increased binding affinity is correlated with a lower Kd.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world-wide-web at sites including ncbi.nlm.nili.gov/BLAST, ebi.ac.uk/Tools/msa/tcoffee, ebi.Ac.Uk/Tools/msa/muscle, mafft.cbrc/alignment/software. See, e.g. Altschul, S. F. et al. (1990). J. Mol. Biol., 215(3):403-410.

A DNA sequence that “encodes” a particular RNA is a DNA sequence that is transcribed into the RNA. A DNA sequence may encode an RNA (an mRNA) that is translated into protein, or an RNA that is not translated into protein (e.g., tRNA, rRNA, or a gRNA; also called “non-coding”RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences.

The term “operably linked,” as used herein, denotes a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter) is a functional linkage that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous.

A cell has been “genetically modified” or “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that include a population of daughter cells containing the transforming DNA. A “clone” is a population of cells derived from a single cell or common ancestor by mitosis. A “cell line” is a clone of a primary cell that is capable of stable growth in vitro for many generations.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation.

The terms “treatment,” “treating,” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, e.g., arresting its development; or (c) relieving the disease, e.g., causing regression of the disease. The therapeutic agent may be administered before, during or after the onset of disease or injury. The treatment of ongoing disease, where the treatment stabilizes or reduces the undesirable clinical symptoms of the subject, is of particular interest. Such treatment is desirably performed prior to complete loss of function in the affected tissues. The therapy will desirably be administered during the symptomatic stage of the disease, and in some cases after the symptomatic stage of the disease.

The terms “individual” and “subject” are used interchangeably herein and refer to any mammalian subject, e.g., a human. In some cases, a subject for whom diagnosis, treatment, or therapy is desired is a patient.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### T-Cell Receptors

T-cell-based immunotherapy targets include peptide epitopes derived from tumor-associated or tumor-specific proteins, which are presented by molecules of the major histocompatibility complex (MHC). These tumors associated antigens (TAAs) can be peptides derived from all protein classes, such as enzymes, receptors, transcription factors, etc. which are expressed and, as compared to unaltered cells of the same origin, usually up-regulated in cells of the respective tumor. Specific elements of the cellular immune response are capable of specifically recognizing and destroying tumor cells. The isolation of T-cells from tumor-infiltrating cell populations or from peripheral blood suggests that such cells play an important role in natural immune defense against cancer. CD8-positive T-cells in particular, which recognize class I molecules of the major histocompatibility complex (MHC)-bearing peptides of usually 8 to 10 amino acid residues derived from proteins or defective ribosomal products (DRiPs) located in the cytosol, play an important role in this response. The MHC-molecules of the human are also designated as human leukocyte-antigens (HLA).

There are two classes of MHC-molecules, MEW class I and MEW class II. Complexes of peptide and WIC class I are recognized by CD8-positive T-cells bearing the appropriate T-cell receptor (TCR), whereas complexes of peptide and WIC class II molecules are recognized by CD4-positive-helper-T-cells bearing the appropriate TCR. Since both types of response, CD8 and CD4 dependent, contribute jointly and synergistically to the anti-tumor effect, the identification and characterization of tumor-associated antigens and corresponding T-cell receptors is important in the development of cancer immunotherapies such as vaccines and cell therapies.

In the WIC class I dependent immune reaction, peptides not only have to be able to bind to certain WIC class I molecules expressed by tumor cells, they subsequently also have to be recognized by T-cells bearing specific T-cell receptors (TCR). Therefore, TAAs are a starting point for the development of a T-cell based therapy including but not limited to tumor vaccines and cell therapies.

The chains of the T-cell antigen receptor of a T-cell clone are each composed of a unique combination of domains designated variable (V), [diversity (D),] joining (J), and constant (C). In each T-cell clone, the combination of V, D, and J domains of both the alpha and the beta chains or of both the delta and gamma chains participates in antigen recognition in a manner which is uniquely characteristic of that T-cell clone and defines a unique binding site, also known as the idiotype of the T-cell clone. In contrast, the C domain does not participate in antigen binding.

A TCR is a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. The extracellular portion of native heterodimeric αβTCR and γδTCR each contain two polypeptides, each of which has a membrane-proximal constant region, and a membrane-distal variable region. Each of the constant and variable regions include an intra-chain disulfide bond. The variable regions contain the highly polymorphic loops analogous to the complementarity determining regions (CDRs), also known as hypervariable regions, of antibodies. In some embodiments, the variable regions of both the TCRα and TCRβ chain each have three CDRs, numbered CDR1, CDR2, and CDR3 in the direction from the amino terminal end to the carboxy terminal end. CDR3 is the main CDR responsible for recognizing processed antigen. In some embodiments, the TCRβ CDR3 has been recognized as more structurally diverse than the other CDRs.

The techniques for determining CDRs are generally known in the art. In some embodiments, the CDRs can be determined by approaches based on cross-species sequence variability. In some embodiments, the CDRs can be determined by approaches based on crystallographic studies of antigen-antibody complexes. In addition, combinations of these approaches are sometimes used in the art to determine CDRs. In certain embodiments, CDRs can be determined using sequence-based prediction tools. Such tools are generally available in the art. In one exemplary embodiment, the CDRs were determined using the Loupe V(D)J Browser provided by 10× Genomics® (Pleasanton, Calif.). For instance, in one embodiment, the single cell TCR sequencing of epitope reactive T-cell population can be conducted using the 10× Genomics® platform. Then the sequence can be processed using the Loupe V(D)J Browser to identify the clonotypes, V(D)J genes, and the CDR motifs, etc. In one specific embodiment, the identified CDR motif is a CDR3. In another specific embodiment, the CDR3 from both the TCRα and the TCRβ variable regions are identified. More detailed information about the Loupe V(D)J Browser is available over the world-wide-web at site: support.10xgenomics.com/single-cell-vdj/software/visualization/latest/tutorial-clonotypes, which is herein incorporated by reference in its entirety.

The use of TCR gene therapy overcomes a number of current hurdles. It allows equipping patients' own T-cells with desired specificities and generation of sufficient numbers of T cells in a short period of time, avoiding their exhaustion. The TCR can be transduced into potent T cells (e.g. central memory T cells or T cells with stem cell characteristics), which may ensure better persistence and function upon transfer. TCR-engineered T cells can be infused into cancer patients rendered lymphopenic by chemotherapy or irradiation, allowing efficient engraftment but inhibiting immune suppression. The present description provides novel TCRs specific for the peptide SLYKGLLSV (SEQ ID NO: 1) found in a cancer-specific isoform of RAD54B, respective recombinant TCR constructs, nucleic acids, vectors and host cells; and methods of using such molecules in the treatment of cancer.

In one aspect, provided herein is an antigen-binding construct comprising i) a TCRα variable region comprising a complementary determining region (CDR) 3 from the TCRα variable region of SEQ ID NO: 12 or a variant thereof having at least 80% sequence identity to the CDR3 from the TCRα variable region of SEQ ID NO: 12; and ii) a TCRβ variable region comprising a CDR3 from the TCRβ variable region of SEQ ID NO: 14 or a variant thereof having at least 80% sequence identity to the CDR3 from the TCRβ variable region of SEQ ID NO: 14. In some embodiments, the TCRα variable region comprises a CDR3 having the amino acid sequence of SEQ ID NO: 8 or a variant thereof having at least 80% sequence identity to the amino acid sequence of SEQ ID NO: 8; and the TCRβ variable region comprises a CDR3 having the amino acid sequence of SEQ ID NO: 11 or a variant thereof having at least 80% sequence identity to the amino acid sequence of SEQ ID NO: 11. In some embodiments, the antigen-binding construct is specific for the peptide SLYKGLLSV (SEQ ID NO: 1) in a peptide/MHC complex. In some embodiments, the MHC molecule in the peptide/MHC complex is HLA-A*02:01. In some embodiments, the antigen-binding construct is a TCR or antigen-binding derivative or fragment thereof.

The term “specific for” in relation to a given antigen as used herein means that the antigen-binding construct can specifically bind to the antigen when it is presented by HLA, e.g., HLA-A*02, such as HLA-A*02:01. For example, a TCR as an antigen-binding construct can be considered to have antigenic specificity for the antigen if T-cells expressing the TCR and contacted with HLA presenting the antigen secrete at least about 200 pg/ml or more (e.g., 250 pg/ml or more, 300 pg/ml or more, 400 pg/ml or more, 500 pg/ml or more, 600 pg/ml or more, 700 pg/ml or more, 1000 pg ml or more, 2,000 pg/ml or more, 2,500 pg/ml or more, 5,000 pg/ml or more) of interferon γ (IFN-γ) upon co-culture with target cells pulsed with a low concentration of the antigen (e.g., about 10⁻¹¹ mol/1, 10⁻¹⁰ mol/1, 10⁻⁹ mol/1, 10⁻⁸ mol/1, 10⁻⁷ mol/1, 10⁻⁶ mol/1, 10⁻⁵ mol/1). Alternatively, or additionally, a TCR may be considered to have antigenic specificity for the antigen if T-cells expressing the TCR secrete at least twice as much I FN-γ as the non-transduced background level of IFN-γ upon co-culture with target cells pulsed with a low concentration of the antigen. Such a specificity as described above can, for example, be analyzed by ELISA.

In some embodiments, according to any of the antigen-binding constructs described herein, the antigen-binding construct selectively binds to the peptide SLYKGLLSV (SEQ ID NO: 1) in a peptide/MHC complex. In some embodiments, the MHC molecule in the peptide/MHC complex is HLA-A*02:01. The term “selectively recognizes/binds” is understood to refer to the property of an antigen-binding construct, such as a TCR, to recognize or bind to one specific epitope and show no or substantially no cross-reactivity to another epitope.

In some embodiments, according to any of the antigen-binding constructs described herein, iii) the TCRα variable region further comprises a CDR1 from the TCRα variable region of SEQ ID NO: 12 or a variant thereof having at least 80% sequence identity to the CDR1 from the TCRα variable region of SEQ ID NO: 12; iv) the TCRα variable region further comprises a CDR2 from the TCRα variable region of SEQ ID NO: 12 or a variant thereof having at least 80% sequence identity to the CDR2 from the TCRα variable region of SEQ ID NO: 12; v) the TCRβ variable region further comprises a CDR1 from the TCRβ variable region of SEQ ID NO: 14 or a variant thereof having at least 80% sequence identity to the CDR1 from the TCRβ variable region of SEQ ID NO: 14; and/or vi) the TCRβ variable region further comprises a CDR2 from the TCRβ variable region of SEQ ID NO: 14 or a variant thereof having at least 80% sequence identity to the CDR2 from the TCRβ variable region of SEQ ID NO: 14.

In some embodiments, according to any of the antigen-binding constructs described herein, iii) the TCRα variable region further comprises a CDR1 from the TCRα variable region of SEQ ID NO: 12 or a variant thereof having at least 80% sequence identity to the CDR1 from the TCRα variable region of SEQ ID NO: 12; iv) the TCRα variable region further comprises a CDR2 from the TCRα variable region of SEQ ID NO: 12 or a variant thereof having at least 80% sequence identity to the CDR2 from the TCRα variable region of SEQ ID NO: 12; v) the TCRβ variable region further comprises a CDR1 from the TCRβ variable region of SEQ ID NO: 14 or a variant thereof having at least 80% sequence identity to the CDR1 from the TCRβ variable region of SEQ ID NO: 14; and vi) the TCRβ variable region further comprises a CDR2 from the TCRβ variable region of SEQ ID NO: 14 or a variant thereof having at least 80% sequence identity to the CDR2 from the TCRβ variable region of SEQ ID NO: 14.

Thus, in some embodiments, provided herein is an antigen-binding construct comprising a) a TCRα variable region comprising i) a CDR1 from the TCRα variable region of SEQ ID NO: 12 or a variant thereof having at least 80% sequence identity to the CDR1 from the TCRα variable region of SEQ ID NO: 12; ii) a CDR2 from the TCRα variable region of SEQ ID NO: 12 or a variant thereof having at least 80% sequence identity to the CDR2 from the TCRα variable region of SEQ ID NO: 12; and iii) a CDR3 from the TCRα variable region of SEQ ID NO: 12 or a variant thereof having at least 80% sequence identity to the CDR3 from the TCRα variable region of SEQ ID NO: 12; and b) a TCRβ variable region comprising i) a CDR1 from the TCRβ variable region of SEQ ID NO: 14 or a variant thereof having at least 80% sequence identity to the CDR1 from the TCRβ variable region of SEQ ID NO: 14; ii) a CDR2 from the TCRβ variable region of SEQ ID NO: 14 or a variant thereof having at least 80% sequence identity to the CDR2 from the TCRβ variable region of SEQ ID NO: 14; and iii) a CDR3 from the TCRβ variable region of SEQ ID NO: 14 or a variant thereof having at least 80% sequence identity to the CDR3 from the TCRβ variable region of SEQ ID NO: 14. In some embodiments, the TCRα variable region comprises a CDR3 having the amino acid sequence of SEQ ID NO: 8 or a variant thereof having at least 80% sequence identity to the amino acid sequence of SEQ ID NO: 8; and the TCRβ variable region comprises a CDR3 having the amino acid sequence of SEQ ID NO: 11 or a variant thereof having at least 80% sequence identity to the amino acid sequence of SEQ ID NO: 11. In some embodiments, the antigen-binding construct is specific for the peptide SLYKGLLSV (SEQ ID NO: 1) in a peptide/WIC complex. In some embodiments, the WIC molecule in the peptide/MHC complex is HLA-A*02:01. In some embodiments, the antigen-binding construct is a TCR or antigen-binding derivative or fragment thereof.

In some embodiments, according to any of the antigen-binding constructs described herein, the TCRα variable region comprises the amino acid sequence of SEQ ID NO: 12 or a variant thereof having at least 80% sequence identity to the amino acid sequence of SEQ ID NO: 12 and/or the TCRβ variable region comprises the amino acid sequence of SEQ ID NO: 14 or a variant thereof having at least 80% sequence identity to the amino acid sequence of SEQ ID NO: 14. In some embodiments, the TCRα variable region comprises the amino acid sequence of SEQ ID NO: 12 or a variant thereof having at least 80% sequence identity to the amino acid sequence of SEQ ID NO: 12 and the TCRβ variable region comprises the amino acid sequence of SEQ ID NO: 14 or a variant thereof having at least 80% sequence identity to the amino acid sequence of SEQ ID NO: 14.

Thus, in some embodiments, provided herein is an antigen-binding construct comprising a TCRα variable region comprising the amino acid sequence of SEQ ID NO: 12 or a variant thereof having at least 80% sequence identity to the amino acid sequence of SEQ ID NO: 12 and a TCRβ variable region comprising the amino acid sequence of SEQ ID NO: 14 or a variant thereof having at least 80% sequence identity to the amino acid sequence of SEQ ID NO: 14. In some embodiments, the antigen-binding construct is specific for the peptide SLYKGLLSV (SEQ ID NO: 1) in a peptide/MHC complex. In some embodiments, the MHC molecule in the peptide/MHC complex is HLA-A*02:01. In some embodiments, the antigen-binding construct is a TCR or antigen-binding derivative or fragment thereof.

In some embodiments, according to any of the antigen-binding constructs described herein, the construct further comprises a TCRα constant region and/or a TCRβ constant region. In some embodiments, the TCRα constant region comprises the amino acid sequence of SEQ ID NO: 13 or a variant thereof having at least 80% sequence identity to the amino acid sequence of SEQ ID NO: 13 and/or the TCRβ constant region comprises the amino acid sequence of SEQ ID NO: 15 or a variant thereof having at least 80% sequence identity to the amino acid sequence of SEQ ID NO: 15. In other embodiments, the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

In some embodiments, according to any of the antigen-binding constructs described herein, the construct is a multimer comprising i) a first polypeptide comprising the TCRα variable region, and ii) a second polypeptide comprising the TCRβ variable region. In some embodiments, the first polypeptide further comprises any other TCRα sequences present in the antigen-binding construct and/or the second polypeptide further comprises any other TCRβ sequences present in the antigen-binding construct. In some embodiments, the first polypeptide comprises the amino acid sequence of SEQ ID NO: 2 or a variant thereof having at least 80% sequence identity to the amino acid sequence of SEQ ID NO: 2 and/or the second polypeptide comprises the amino acid sequence of SEQ ID NO: 3 or a variant thereof having at least 80% sequence identity to the amino acid sequence of SEQ ID NO: 3. In some embodiments, the first polypeptide comprises the amino acid sequence of SEQ ID NO: 16 or a variant thereof having at least 80% sequence identity to the amino acid sequence of SEQ ID NO: 16 and/or the second polypeptide comprises the amino acid sequence of SEQ ID NO: 17 or a variant thereof having at least 80% sequence identity to the amino acid sequence of SEQ ID NO: 17.

In some embodiments, according to any of the antigen-binding constructs described herein, the construct is a single chain polypeptide.

In some embodiments, according to any of the antigen-binding constructs described herein, T-cells expressing the construct are capable of inducing an immune response in a subject when contacted with cells presenting the peptide SLYKGLLSV (SEQ ID NO: 1) in a peptide/MHC complex. In some embodiments, the WIC molecule in the peptide/MHC complex is HLA-A*02:01. In some embodiments, the immune response is characterized by an increase in IFN-γ levels.

In some embodiments, provided herein is a TCR or antigen-binding derivative or fragment thereof comprising i) a TCRα variable region comprising a complementary determining region (CDR) 3 from the TCRα variable region of SEQ ID NO: 12 or a variant thereof having at least 80% sequence identity to the CDR3 from the TCRα variable region of SEQ ID NO: 12; and ii) a TCRβ variable region comprising a CDR3 from the TCRβ variable region of SEQ ID NO: 14 or a variant thereof having at least 80% sequence identity to the CDR3 from the TCRβ variable region of SEQ ID NO: 14. In some embodiments, the TCRα variable region comprises a CDR3 having the amino acid sequence of SEQ ID NO: 8 or a variant thereof having at least 80% sequence identity to the amino acid sequence of SEQ ID NO: 8; and the TCRβ variable region comprises a CDR3 having the amino acid sequence of SEQ ID NO: 11 or a variant thereof having at least 80% sequence identity to the amino acid sequence of SEQ ID NO: 11. In some embodiments, the TCR is specific for the peptide SLYKGLLSV (SEQ ID NO: 1) in a peptide/MHC complex. In some embodiments, the MHC molecule in the peptide/MHC complex is HLA-A*02:01.

In some embodiments, according to any of the TCRs or antigen-binding derivatives or fragments thereof described herein, iii) the TCRα variable region further comprises a CDR1 from the TCRα variable region of SEQ ID NO: 12 or a variant thereof having at least 80% sequence identity to the CDR1 from the TCRα variable region of SEQ ID NO: 12; iv) the TCRα variable region further comprises a CDR2 from the TCRα variable region of SEQ ID NO: 12 or a variant thereof having at least 80% sequence identity to the CDR2 from the TCRα variable region of SEQ ID NO: 12; v) the TCRβ variable region further comprises a CDR1 from the TCRβ variable region of SEQ ID NO: 14 or a variant thereof having at least 80% sequence identity to the CDR1 from the TCRβ variable region of SEQ ID NO: 14; and/or vi) the TCRβ variable region further comprises a CDR2 from the TCRβ variable region of SEQ ID NO: 14 or a variant thereof having at least 80% sequence identity to the CDR2 from the TCRβ variable region of SEQ ID NO: 14.

In some embodiments, according to any of the TCRs or antigen-binding derivatives or fragments thereof described herein, iii) the TCRα variable region further comprises a CDR1 from the TCRα variable region of SEQ ID NO: 12 or a variant thereof having at least 80% sequence identity to the CDR1 from the TCRα variable region of SEQ ID NO: 12; iv) the TCRα variable region further comprises a CDR2 from the TCRα variable region of SEQ ID NO: 12 or a variant thereof having at least 80% sequence identity to the CDR2 from the TCRα variable region of SEQ ID NO: 12; v) the TCRβ variable region further comprises a CDR1 from the TCRβ variable region of SEQ ID NO: 14 or a variant thereof having at least 80% sequence identity to the CDR1 from the TCRβ variable region of SEQ ID NO: 14; and vi) the TCRβ variable region further comprises a CDR2 from the TCRβ variable region of SEQ ID NO: 14 or a variant thereof having at least 80% sequence identity to the CDR2 from the TCRβ variable region of SEQ ID NO: 14.

Thus, in some embodiments, provided herein is a TCR or antigen-binding derivative or fragment thereof comprising a) a TCRα variable region comprising i) a CDR1 from the TCRα variable region of SEQ ID NO: 12 or a variant thereof having at least 80% sequence identity to the CDR1 from the TCRα variable region of SEQ ID NO: 12; ii) a CDR2 from the TCRα variable region of SEQ ID NO: 12 or a variant thereof having at least 80% sequence identity to the CDR2 from the TCRα variable region of SEQ ID NO: 12; and iii) a CDR3 from the TCRα variable region of SEQ ID NO: 12 or a variant thereof having at least 80% sequence identity to the CDR3 from the TCRα variable region of SEQ ID NO: 12; and b) a TCRβ variable region comprising i) a CDR1 from the TCRβ variable region of SEQ ID NO: 14 or a variant thereof having at least 80% sequence identity to the CDR1 from the TCRβ variable region of SEQ ID NO: 14; ii) a CDR2 from the TCRβ variable region of SEQ ID NO: 14 or a variant thereof having at least 80% sequence identity to the CDR2 from the TCRβ variable region of SEQ ID NO: 14; and iii) a CDR3 from the TCRβ variable region of SEQ ID NO: 14 or a variant thereof having at least 80% sequence identity to the CDR3 from the TCRβ variable region of SEQ ID NO: 14. In some embodiments, the TCRα variable region comprises a CDR3 having the amino acid sequence of SEQ ID NO: 8 or a variant thereof having at least 80% sequence identity to the amino acid sequence of SEQ ID NO: 8; and the TCRβ variable region comprises a CDR3 having the amino acid sequence of SEQ ID NO: 11 or a variant thereof having at least 80% sequence identity to the amino acid sequence of SEQ ID NO: 11. In some embodiments, the TCR is specific for the peptide SLYKGLLSV (SEQ ID NO: 1) in a peptide/MHC complex. In some embodiments, the MHC molecule in the peptide/MHC complex is HLA-A*02:01.

In some embodiments, according to any of the TCRs or antigen-binding derivatives or fragments thereof described herein, the TCRα variable region comprises the amino acid sequence of SEQ ID NO: 12 or a variant thereof having at least 80% sequence identity to the amino acid sequence of SEQ ID NO: 12 and/or the TCRβ variable region comprises the amino acid sequence of SEQ ID NO: 14 or a variant thereof having at least 80% sequence identity to the amino acid sequence of SEQ ID NO: 14. In some embodiments, the TCRα variable region comprises the amino acid sequence of SEQ ID NO: 12 or a variant thereof having at least 80% sequence identity to the amino acid sequence of SEQ ID NO: 12 and the TCRβ variable region comprises the amino acid sequence of SEQ ID NO: 14 or a variant thereof having at least 80% sequence identity to the amino acid sequence of SEQ ID NO: 14.

Thus, in some embodiments, provided herein is a TCR comprising a TCRα variable region comprising the amino acid sequence of SEQ ID NO: 12 or a variant thereof having at least 80% sequence identity to the amino acid sequence of SEQ ID NO: 12 and a TCRβ variable region comprising the amino acid sequence of SEQ ID NO: 14 or a variant thereof having at least 80% sequence identity to the amino acid sequence of SEQ ID NO: 14. In some embodiments, the TCR is specific for the peptide SLYKGLLSV (SEQ ID NO: 1) in a peptide/MHC complex. In some embodiments, the WIC molecule in the peptide/MHC complex is HLA-A*02:01.

In some embodiments, according to any of the TCRs or antigen-binding derivatives or fragments thereof described herein, the variable regions of the TCR are modified, for example, by the introduction of one or more mutations to optimize the TCR stability and/or to enhance TCR chain pairing.

In some embodiments, according to any of the TCRs or antigen-binding derivatives or fragments thereof described herein, the TCR or antigen-binding derivative or fragment thereof further comprises a TCRα constant region and/or a TCRβ constant region. In some embodiments, the TCRα constant region and/or the TCRβ constant region are derived from human. In some embodiments, the TCRα constant region comprises the amino acid sequence of SEQ ID NO: 13 or a variant thereof having at least 80% sequence identity to the amino acid sequence of SEQ ID NO: 13 and/or the TCRβ constant region comprises the amino acid sequence of SEQ ID NO: 15 or a variant thereof having at least 80% sequence identity to the amino acid sequence of SEQ ID NO: 15. In other embodiments, the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

In some embodiments, according to any of the TCRs or antigen-binding derivatives or fragments thereof described herein, the constant regions of the TCR are modified, for example, by the introduction of heterologous sequences, e.g. mouse sequences, that increase TCR expression and stability.

In some embodiments, according to any of the TCRs or antigen-binding derivatives or fragments thereof described herein, the TCR or antigen-binding derivative or fragment thereof comprises an α chain subunit comprising the amino acid sequence of SEQ ID NO: 2 or a variant thereof having at least 80% sequence identity to the amino acid sequence of SEQ ID NO: 2 and/or a β chain subunit comprising the amino acid sequence of SEQ ID NO: 3 or a variant thereof having at least 80% sequence identity to the amino acid sequence of SEQ ID NO: 3. In other embodiments, according to any of the TCRs or antigen-binding derivatives or fragments thereof described herein, the TCR or antigen-binding derivative or fragment thereof comprises an α chain subunit comprising the amino acid sequence of SEQ ID NO: 16 or a variant thereof having at least 80% sequence identity to the amino acid sequence of SEQ ID NO: 16 and/or a β chain subunit comprising the amino acid sequence of SEQ ID NO: 17 or a variant thereof having at least 80% sequence identity to the amino acid sequence of SEQ ID NO: 17.

In some embodiments, according to any of the TCRs or antigen-binding derivatives or fragments thereof described herein, the TCR is a human TCR, which is understood as comprising human TCR sequences.

In some embodiments, according to any of the TCRs or antigen-binding derivatives or fragments thereof described herein, the TCR is a chimeric TCR comprising sequences from multiple species. For example, in some embodiments, the TCR comprises an α chain subunit comprising a human α chain variable region and, for example, a murine α chain constant region.

In some embodiments, according to any of the TCRs or antigen-binding derivatives or fragments thereof described herein, the TCR is a single chain TCR (scTCR) comprising in one polypeptide chain a full or partial alpha chain sequence and a full or partial beta chain sequence. In some embodiments, the full or partial alpha chain sequence is connected to the full or partial beta chain sequence via a peptide linker.

As used in relation to an antigen-binding construct provided herein, a TCR is a moiety comprising a TCR alpha variable region and a TCR beta variable region, wherein the moiety is capable of recognizing an antigen and configured such that when associated with a T-cell (e.g., CD8+ T-cell) it allows for activation of the T-cell upon antigen binding. They may be αβ heterodimers or may be in single chain format. For use in adoptive therapy, an αβ heterodimeric TCR may, for example, be employed as full-length chains including extracellular, transmembrane, and cytoplasmic domains. In some embodiments, an introduced disulfide bond between residues of the respective constant domains may be present. In some embodiments, an antigen-binding construct provided herein is a TCR, or fragment or derivative thereof, such as a human TCR, or fragment or derivative thereof. In some embodiments, a portion of the TCR sequence is of artificial origin or derived from other species. In some embodiments, the TCR is a chimeric TCR, e.g., a chimeric TCR derived from human origin with murine sequences in the constant domains. In some embodiments, a TCR according to any of the embodiments described herein comprises murine sequences in the extracellular part of their constant domains.

### Nucleic Acids

In another aspect, provided herein is nucleic acid encoding an antigen-binding construct (such as a TCR or antigen-binding derivative or fragment thereof) according to any of the embodiments described herein. In some embodiments, such a nucleic acid is a vector (e.g., a recombinant expression vector).

In some embodiments, provided herein is nucleic acid encoding an antigen-binding construct (such as a TCR or antigen-binding derivative or fragment thereof) comprising a) a TCRα variable region comprising i) a CDR1 from the TCRα variable region of SEQ ID NO: 12 or a variant thereof having at least 80% sequence identity to the CDR1 from the TCRα variable region of SEQ ID NO: 12; ii) a CDR2 from the TCRα variable region of SEQ ID NO: 12 or a variant thereof having at least 80% sequence identity to the CDR2 from the TCRα variable region of SEQ ID NO: 12; and/or iii) a CDR3 from the TCRα variable region of SEQ ID NO: 12 or a variant thereof having at least 80% sequence identity to the CDR3 from the TCRα variable region of SEQ ID NO: 12; and/or b) a TCRβ variable region comprising i) a CDR1 from the TCRβ variable region of SEQ ID NO: 14 or a variant thereof having at least 80% sequence identity to the CDR1 from the TCRβ variable region of SEQ ID NO: 14; ii) a CDR2 from the TCRβ variable region of SEQ ID NO: 14 or a variant thereof having at least 80% sequence identity to the CDR2 from the TCRβ variable region of SEQ ID NO: 14; and/or iii) a CDR3 from the TCRβ variable region of SEQ ID NO: 14 or a variant thereof having at least 80% sequence identity to the CDR3 from the TCRβ variable region of SEQ ID NO: 14. In some embodiments, the TCRα variable region comprises a CDR3 having the amino acid sequence of SEQ ID NO: 8 or a variant thereof having at least 80% sequence identity to the amino acid sequence of SEQ ID NO: 8; and the TCRβ variable region comprises a CDR3 having the amino acid sequence of SEQ ID NO: 11 or a variant thereof having at least 80% sequence identity to the amino acid sequence of SEQ ID NO: 11. In some embodiments, the antigen-binding construct is specific for the peptide SLYKGLLSV (SEQ ID NO: 1) in a peptide/MHC complex. In some embodiments, the MHC molecule in the peptide/MHC complex is HLA-A*02:01. In some embodiments, the antigen-binding construct is a TCR or antigen-binding derivative or fragment thereof.

In some embodiments, provided herein is nucleic acid encoding an antigen-binding construct comprising a TCRα variable region comprising the amino acid sequence of SEQ ID NO: 12 or a variant thereof having at least 80% sequence identity to the amino acid sequence of SEQ ID NO: 12 and/or a TCRβ variable region comprising the amino acid sequence of SEQ ID NO: 14 or a variant thereof having at least 80% sequence identity to the amino acid sequence of SEQ ID NO: 14. In some embodiments, the antigen-binding construct is specific for the peptide SLYKGLLSV (SEQ ID NO: 1) in a peptide/MHC complex. In some embodiments, the MHC molecule in the peptide/MHC complex is HLA-A*02:01. In some embodiments, the antigen-binding construct is a TCR or antigen-binding derivative or fragment thereof.

In some embodiments, according to any of the nucleic acids encoding an antigen-binding construct described herein, the construct further comprises a TCRα constant region and/or a TCRβ constant region. In some embodiments, the TCRα constant region comprises the amino acid sequence of SEQ ID NO: 13 or a variant thereof having at least 80% sequence identity to the amino acid sequence of SEQ ID NO: 13 and/or the TCRβ constant region comprises the amino acid sequence of SEQ ID NO: 15 or a variant thereof having at least 80% sequence identity to the amino acid sequence of SEQ ID NO: 15. In other embodiments, the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

In some embodiments, according to any of the nucleic acids encoding an antigen-binding construct described herein, the construct is a multimer comprising i) a first polypeptide comprising the TCRα variable region, and ii) a second polypeptide comprising the TCRβ variable region. In some embodiments, the first polypeptide further comprises any other TCRα sequences present in the antigen-binding construct and/or the second polypeptide further comprises any other TCRβ sequences present in the antigen-binding construct. In some embodiments, the nucleic acid comprises a first coding sequence encoding the first polypeptide, the first coding sequence comprising the nucleotide sequence of SEQ ID NO: 4 or a variant thereof having at least 80% sequence identity to the amino acid sequence of SEQ ID NO: 4; and/or a second coding sequence encoding the second polypeptide, the second coding sequence comprising the amino acid sequence of SEQ ID NO: 5 or a variant thereof having at least 80% sequence identity to the amino acid sequence of SEQ ID NO: 5. In other embodiments, the nucleic acid comprises a first coding sequence encoding the first polypeptide, the first coding sequence comprising the nucleotide sequence of SEQ ID NO: 18 or a variant thereof having at least 80% sequence identity to the amino acid sequence of SEQ ID NO: 18; and/or a second coding sequence encoding the second polypeptide, the second coding sequence comprising the amino acid sequence of SEQ ID NO: 19 or a variant thereof having at least 80% sequence identity to the amino acid sequence of SEQ ID NO: 19. In some embodiments, the nucleic acid encoding the antigen-binding construct comprises a single nucleic acid molecule comprising the first and second coding sequences. In some embodiments, the nucleic acid encoding the antigen-binding construct comprises distinct nucleic acid molecules comprising the first and second coding sequences.

In some embodiments, according to any of the nucleic acids encoding an antigen-binding construct described herein, the construct is a single chain polypeptide.

In some embodiments, provided herein is nucleic acid encoding a TCR comprising i) a first coding sequence encoding an α chain subunit, the first coding sequence comprising the nucleotide sequence of SEQ ID NO: 4 or a variant thereof having at least 80% sequence identity to the amino acid sequence of SEQ ID NO: 4; and/or ii) a second coding sequence encoding a (3 chain subunit, the second coding sequence comprising the nucleotide sequence of SEQ ID NO: 5 or a variant thereof having at least 80% sequence identity to the amino acid sequence of SEQ ID NO: 5. In other embodiments, provided herein is nucleic acid encoding a TCR comprising i) a first coding sequence encoding an α chain subunit, the first coding sequence comprising the nucleotide sequence of SEQ ID NO: 18 or a variant thereof having at least 80% sequence identity to the amino acid sequence of SEQ ID NO: 18; and/or ii) a second coding sequence encoding a β chain subunit, the second coding sequence comprising the nucleotide sequence of SEQ ID NO: 19 or a variant thereof having at least 80% sequence identity to the amino acid sequence of SEQ ID NO: 19. In some embodiments, the nucleic acid encoding the TCR comprises a single nucleic acid molecule comprising the first and second coding sequences. In some embodiments, the nucleic acid encoding the TCR comprises distinct nucleic acid molecules comprising the first and second coding sequences. In some embodiments, the TCR is specific for the peptide SLYKGLLSV (SEQ ID NO: 1) in a peptide/MHC complex. In some embodiments, the MHC molecule in the peptide/MHC complex is HLA-A*02:01.

Expression vectors contemplated include, but are not limited to, viral vectors based on vaccinia virus, poliovirus, adenovirus, AAV, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors can be used so long as they are compatible with the cell.

In some embodiments, a vector according to any of the embodiments described herein comprises one or more transcription and/or translation control elements. Depending on the host/vector system utilized, any of a number of suitable transcription and translation control elements, including constitutive and inducible promoters, transcription enhancer elements, transcription terminators, etc. can be used in the expression vector.

In some embodiments, a vector according to any of the embodiments described herein comprises a ribosome binding site for translation initiation and a transcription terminator. In some embodiments, the vector comprises appropriate sequences for amplifying expression. In some embodiments, the vector comprises nucleotide sequences encoding non-native tags (e.g., histidine tags, hemagglutinin tags, green fluorescent proteins, etc.) that are fused to nucleotide sequences encoding a polypeptide of interest (e.g., an antigen-binding construct), thus allowing for expression of fusion proteins comprising the tags.

In some embodiments, according to any of the vectors described herein comprising a promoter, the promoter is an inducible promoter (e.g., a heat shock promoter, tetracycline-regulated promoter, steroid-regulated promoter, metal-regulated promoter, estrogen receptor-regulated promoter, etc.) or a constitutive promoter (e.g., CMV promoter, UBC promoter, etc.). In some embodiments, the promoter is a spatially restricted and/or temporally restricted promoter (e.g., a tissue specific promoter, a cell type specific promoter, etc.).

### Host Cells

In one aspect, provided herein is a host cell comprising or capable of expressing an antigen-binding construct (such as a TCR or antigen-binding derivative or fragment thereof) according to any of the embodiments described herein. In some embodiments, the host cell comprises a nucleic acid or a vector as described herein, such as a heterologous nucleic acid or vector. In some embodiments, the host cell is a mammalian cell, such as a human cell. The host cell can be a cultured cell or a primary cell, i.e., isolated directly from an organism, e.g., a human. The host cell can be an adherent cell or a suspended cell, i.e., a cell that grows in suspension. In some embodiments, the host cell is a peripheral blood leukocyte (PBL) or a peripheral blood mononuclear cell (PBMC). In some embodiments, the host cell is a T-cell. The T-cell can be any T-cell, such as a cultured T-cell, e.g., a primary T-cell, or a T-cell from a cultured T-cell line, e.g., Jurkat, SupT1, etc., or a T-cell obtained from a mammal, such as a T-cell or T-cell precursor from a human patient. If obtained from a mammal, the T-cell can be obtained from numerous sources, including but not limited to blood, bone marrow, lymph node, the thymus, or other tissues or fluids. T-cells can also be enriched for or purified. The T-cell can be any type of T-cell and can be of any developmental stage, including but not limited to, CD4-positive and/or CD8-positive, CD4-positive helper T-cells, e.g., Th1 and Th2 cells, CD8-positive T-cells (e.g., cytotoxic T-cells), tumor infiltrating cells (TILs), memory T-cells, naive T-cells, and the like. In some embodiments, the T-cell is a CD8-positive T-cell or a CD4-positive T-cell.

In some embodiments, provided herein is a host cell comprising or capable of expressing an antigen-binding construct (such as a TCR or antigen-binding derivative or fragment thereof) comprising a) a TCRα variable region comprising i) a CDR1 from the TCRα variable region of SEQ ID NO: 12 or a variant thereof having at least 80% sequence identity to the CDR1 from the TCRα variable region of SEQ ID NO: 12; ii) a CDR2 from the TCRα variable region of SEQ ID NO: 12 or a variant thereof having at least 80% sequence identity to the CDR2 from the TCRα variable region of SEQ ID NO: 12; and/or iii) a CDR3 from the TCRα variable region of SEQ ID NO: 12 or a variant thereof having at least 80% sequence identity to the CDR3 from the TCRα variable region of SEQ ID NO: 12; and/or b) a TCRβ variable region comprising i) a CDR1 from the TCRβ variable region of SEQ ID NO: 14 or a variant thereof having at least 80% sequence identity to the CDR1 from the TCRβ variable region of SEQ ID NO: 14; ii) a CDR2 from the TCRβ variable region of SEQ ID NO: 14 or a variant thereof having at least 80% sequence identity to the CDR2 from the TCRβ variable region of SEQ ID NO: 14; and/or iii) a CDR3 from the TCRβ variable region of SEQ ID NO: 14 or a variant thereof having at least 80% sequence identity to the CDR3 from the TCRβ variable region of SEQ ID NO: 14. In some embodiments, the TCRα variable region comprises a CDR3 having the amino acid sequence of SEQ ID NO: 8 or a variant thereof having at least 80% sequence identity to the amino acid sequence of SEQ ID NO: 8; and/or the TCRβ variable region comprises a CDR3 having the amino acid sequence of SEQ ID NO: 11 or a variant thereof having at least 80% sequence identity to the amino acid sequence of SEQ ID NO: 11. In some embodiments, the antigen-binding construct is specific for the peptide SLYKGLLSV (SEQ ID NO: 1) in a peptide/MHC complex. In some embodiments, the MHC molecule in the peptide/MHC complex is HLA-A*02:01. In some embodiments, the antigen-binding construct is a TCR or antigen-binding derivative or fragment thereof. In some embodiments, the host cell comprises nucleic acid encoding the antigen-binding construct. In some embodiments, the nucleic acid encoding the antigen-binding construct is heterologous to the host cell. In some embodiments, the host cell is a T-cell, such as a human T-cell. In some embodiments, the T-cell is a CD8+ T-cell.

In some embodiments, provided herein is a host cell comprising or capable of expressing an antigen-binding construct (such as a TCR or antigen-binding derivative or fragment thereof) comprising a TCRα variable region comprising the amino acid sequence of SEQ ID NO: 12 or a variant thereof having at least 80% sequence identity to the amino acid sequence of SEQ ID NO: 12 and/or a TCRβ variable region comprising the amino acid sequence of SEQ ID NO: 14 or a variant thereof having at least 80% sequence identity to the amino acid sequence of SEQ ID NO: 14. In some embodiments, the antigen-binding construct is specific for the peptide SLYKGLLSV (SEQ ID NO: 1) in a peptide/MHC complex. In some embodiments, the MHC molecule in the peptide/MHC complex is HLA-A*02:01. In some embodiments, the antigen-binding construct is a TCR or antigen-binding derivative or fragment thereof. In some embodiments, the host cell comprises nucleic acid encoding the antigen-binding construct. In some embodiments, the nucleic acid encoding the antigen-binding construct is heterologous to the host cell. In some embodiments, the host cell is a T-cell, such as a human T-cell. In some embodiments, the T-cell is a CD8+ T-cell.

In some embodiments, according to any of the host cells comprising or capable of expressing an antigen-binding construct described herein, the construct further comprises a TCRα constant region and/or a TCRβ constant region. In some embodiments, the TCRα constant region comprises the amino acid sequence of SEQ ID NO: 13 or a variant thereof having at least 80% sequence identity to the amino acid sequence of SEQ ID NO: 13 and/or the TCRβ constant region comprises the amino acid sequence of SEQ ID NO: 15 or a variant thereof having at least 80% sequence identity to the amino acid sequence of SEQ ID NO: 15. In other embodiments, the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

In some embodiments, according to any of the host cells comprising or capable of expressing an antigen-binding construct described herein, the construct is a multimer comprising i) a first polypeptide comprising the TCRα variable region, and ii) a second polypeptide comprising the TCRβ variable region. In some embodiments, the first polypeptide further comprises any other TCRα sequences present in the antigen-binding construct and/or the second polypeptide further comprises any other TCRβ sequences present in the antigen-binding construct. In some embodiments, the first polypeptide comprises the amino acid sequence of SEQ ID NO: 2 or a variant thereof having at least 80% sequence identity to the amino acid sequence of SEQ ID NO: 2 and/or the second polypeptide comprises the amino acid sequence of SEQ ID NO: 3 or a variant thereof having at least 80% sequence identity to the amino acid sequence of SEQ ID NO: 3. In other embodiments, the first polypeptide comprises the amino acid sequence of SEQ ID NO: 16 or a variant thereof having at least 80% sequence identity to the amino acid sequence of SEQ ID NO: 16 and/or the second polypeptide comprises the amino acid sequence of SEQ ID NO: 17 or a variant thereof having at least 80% sequence identity to the amino acid sequence of SEQ ID NO: 17.

In some embodiments, according to any of the host cells comprising or capable of expressing an antigen-binding construct described herein, the construct is a single chain polypeptide.

In some embodiments, according to any of the host cells comprising or capable of expressing an antigen-binding construct described herein, the host cell is a T-cell, such as a human T-cell. In some embodiments, the T-cell is a CD8+ T-cell.

In some embodiments, according to any of the host cells comprising or capable of expressing an antigen-binding construct described herein, the host cell is a T-cell (e.g., a CD8+ T-cell), and the T-cell is capable of inducing an immune response in a subject when contacted with cells presenting the peptide SLYKGLLSV (SEQ ID NO: 1) in a peptide/MHC complex. In some embodiments, the MHC molecule in the peptide/MHC complex is HLA-A*02:01. In some embodiments, the immune response is characterized by an increase in IFN-γ levels.

The present disclosure further provides progeny of an engineered cell, where the progeny can include the same heterologous nucleic acid or polypeptide as the engineered cell from which it was derived. The present disclosure further provides, in some embodiments, a composition comprising an engineered cell.

Nucleic acids and vectors as described herein may be provided to the cells using well-developed transfection techniques; see, e.g., Angel, M. et al. (2010). PLoS ONE, 5(7):e11756, and the commercially available TransMessenger® reagents from Qiagen, Stemfect™ RNA Transfection Kit from Stemgent, and TransiT®-mRNA Transfection Kit from Mims Bio. See also Beumer, K. J. et al. (2008). Proc. Natl. Acad. Sci. USA, 105(50):19821-19826. Many vectors, e.g., plasmids, cosmids, minicircles, phage, viruses, etc., useful for transferring nucleic acids into target cells are available. The vectors comprising the nucleic acid(s) may be maintained episomally, e.g., as plasmids, minicircle DNAs, viruses such cytomegalovirus, adenovirus, etc., or they may be integrated into the target cell genome, through homologous recombination or random integration, e.g., retrovirus-derived vectors such as MMLV, HIV-1, ALV, etc.

Vectors may be provided directly to the cells. In other words, the cells are contacted with vectors such that the vectors are taken up by the cells. Methods for contacting cells with nucleic acid vectors that are plasmids, including electroporation, calcium chloride transfection, microinjection, and lipofection are well known in the art. For viral vector delivery, the cells are contacted with viral particles comprising nucleic acid as described herein. Retroviruses, for example, lentiviruses, are particularly suitable to the method of the present disclosure. Commonly used retroviral vectors are “defective”, e.g., unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid encoding the reprogramming factors into packaging cell lines and of collecting the viral particles that are generated by the packaging lines are well known in the art. Nucleic acids can also be introduced by direct micro-injection (e.g., injection of RNA into a zebrafish embryo).

Vectors will generally comprise suitable promoters for driving the expression, that is, transcriptional activation, of the nucleic acid of interest. In other words, the nucleic acid of interest will be operably linked to a promoter. This may include ubiquitously active promoters, for example, the CMV-13-actin promoter, or inducible promoters, such as promoters that are active in particular cell populations or that respond to the presence of drugs such as tetracycline. By transcriptional activation, it is intended that transcription will be increased above basal levels in the target cell by at least about 10-fold (such as by at least about any of 100-fold, 1000-fold, or greater). In addition, vectors may include nucleic acid sequences that code for selectable markers in the target cells.

### Methods

### Methods of Preparing a T-Cell Comprising an Antigen-Binding Construct

In one aspect, provided herein is a method of preparing a T-cell comprising or capable of expressing an antigen-binding construct (such as a TCR or antigen-binding derivative or fragment thereof) according to any of the embodiments described herein, comprising introducing nucleic acid (e.g., heterologous nucleic acid) encoding the antigen-binding construct into an input T-cell (e.g., an input CD8+ T-cell), wherein the antigen-binding construct is capable of being expressed in the input T-cell following introduction of the nucleic acid.

In some embodiments, provided herein is a method of preparing a T-cell comprising or capable of expressing an antigen-binding construct (such as a TCR or antigen-binding derivative or fragment thereof), the method comprising introducing nucleic acid encoding the antigen-binding construct into an input T-cell, wherein the antigen-binding construct comprises a) a TCRα variable region comprising i) a CDR1 from the TCRα variable region of SEQ ID NO: 12 or a variant thereof having at least 80% sequence identity to the CDR1 from the TCRα variable region of SEQ ID NO: 12; ii) a CDR2 from the TCRα variable region of SEQ ID NO: 12 or a variant thereof having at least 80% sequence identity to the CDR2 from the TCRα variable region of SEQ ID NO: 12; and/or iii) a CDR3 from the TCRα variable region of SEQ ID NO: 12 or a variant thereof having at least 80% sequence identity to the CDR3 from the TCRα variable region of SEQ ID NO: 12; and/or b) a TCRβ variable region comprising i) a CDR1 from the TCRβ variable region of SEQ ID NO: 14 or a variant thereof having at least 80% sequence identity to the CDR1 from the TCRβ variable region of SEQ ID NO: 14; ii) a CDR2 from the TCRβ variable region of SEQ ID NO: 14 or a variant thereof having at least 80% sequence identity to the CDR2 from the TCRβ variable region of SEQ ID NO: 14; and/or iii) a CDR3 from the TCRβ variable region of SEQ ID NO: 14 or a variant thereof having at least 80% sequence identity to the CDR3 from the TCRβ variable region of SEQ ID NO: 14, and wherein the antigen-binding construct is capable of being expressed in the input T-cell following introduction of the nucleic acid. In some embodiments, the TCRα variable region comprises a CDR3 having the amino acid sequence of SEQ ID NO: 8 or a variant thereof having at least 80% sequence identity to the amino acid sequence of SEQ ID NO: 8; and/or the TCRβ variable region comprises a CDR3 having the amino acid sequence of SEQ ID NO: 11 or a variant thereof having at least 80% sequence identity to the amino acid sequence of SEQ ID NO: 11. In some embodiments, the antigen-binding construct is specific for the peptide SLYKGLLSV (SEQ ID NO: 1) in a peptide/WIC complex. In some embodiments, the WIC molecule in the peptide/WIC complex is HLA-A*02:01. In some embodiments, the antigen-binding construct is a TCR or antigen-binding derivative or fragment thereof. In some embodiments, the T-cell is human. In some embodiments, the T-cell is a CD8+ T-cell. In some embodiments, the nucleic acid is heterologous to the input T-cell.

In some embodiments, provided herein is a method of preparing a T-cell comprising or capable of expressing an antigen-binding construct (such as a TCR or antigen-binding derivative or fragment thereof), the method comprising introducing nucleic acid encoding the antigen-binding construct into an input T-cell, wherein the antigen-binding construct comprises a TCRα variable region comprising the amino acid sequence of SEQ ID NO: 12 or a variant thereof having at least 80% sequence identity to the amino acid sequence of SEQ ID NO: 12 and/or a TCRβ variable region comprising the amino acid sequence of SEQ ID NO: 14 or a variant thereof having at least 80% sequence identity to the amino acid sequence of SEQ ID NO: 14, and wherein the antigen-binding construct is capable of being expressed in the input T-cell following introduction of the nucleic acid. In some embodiments, the antigen-binding construct is specific for the peptide SLYKGLLSV (SEQ ID NO: 1) in a peptide/WIC complex. In some embodiments, the WIC molecule in the peptide/WIC complex is HLA-A*02:01. In some embodiments, the antigen-binding construct is a TCR or antigen-binding derivative or fragment thereof. In some embodiments, the T-cell is human. In some embodiments, the T-cell is a CD8+ T-cell. In some embodiments, the nucleic acid is heterologous to the input T-cell.

In some embodiments, according to any of the methods of preparing a T-cell described herein, the construct further comprises a TCRα constant region and/or a TCRβ constant region. In some embodiments, the TCRα constant region comprises the amino acid sequence of SEQ ID NO: 13 or a variant thereof having at least 80% sequence identity to the amino acid sequence of SEQ ID NO: 13 and/or the TCRβ constant region comprises the amino acid sequence of SEQ ID NO: 15 or a variant thereof having at least 80% sequence identity to the amino acid sequence of SEQ ID NO: 15. In other embodiments, the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

In some embodiments, according to any of the methods of preparing a T-cell described herein, the construct is a multimer comprising i) a first polypeptide comprising the TCRα variable region, and ii) a second polypeptide comprising the TCRβ variable region. In some embodiments, the first polypeptide further comprises any other TCRα sequences present in the antigen-binding construct and/or the second polypeptide further comprises any other TCRβ sequences present in the antigen-binding construct. In some embodiments, the first polypeptide comprises the amino acid sequence of SEQ ID NO: 2 or a variant thereof having at least 80% sequence identity to the amino acid sequence of SEQ ID NO: 2 and/or the second polypeptide comprises the amino acid sequence of SEQ ID NO: 3 or a variant thereof having at least 80% sequence identity to the amino acid sequence of SEQ ID NO: 3. In some embodiments, the first polypeptide comprises the amino acid sequence of SEQ ID NO: 16 or a variant thereof having at least 80% sequence identity to the amino acid sequence of SEQ ID NO: 16 and/or the second polypeptide comprises the amino acid sequence of SEQ ID NO: 17 or a variant thereof having at least 80% sequence identity to the amino acid sequence of SEQ ID NO: 17.

In some embodiments, according to any of the methods of preparing a T-cell described herein, the construct is a single chain polypeptide.

In some embodiments, according to any of the methods of preparing a T-cell described herein, the T-cell (e.g., CD8+ T-cell) comprising or capable of expressing an antigen-binding construct is capable of inducing an immune response in a subject when contacted with cells presenting the peptide SLYKGLLSV (SEQ ID NO: 1) in a peptide/MHC complex. In some embodiments, the MHC molecule in the peptide/MHC complex is HLA-A*02:01. In some embodiments, the immune response is characterized by an increase in IFN-γ levels.

In another aspect, provided herein are T-cells comprising an antigen-binding construct prepared according to any of the methods described herein. In some embodiments, the T-cell are CD8+ T-cells.

### Methods of Inducing an Immune Response

In one aspect, provided herein is a method of inducing an immune response to an isoform of RAD54B comprising the peptide RAD54B₆₁₈₋₆₂₆ (SLYKGLLSV, SEQ ID NO: 1) in a subject, comprising administering to the subject a T-cell (e.g., CD8+ T-cell) comprising or capable of expressing an antigen-binding construct (such as a TCR or antigen-binding derivative or fragment thereof) according to any of the embodiments described herein. In some embodiments, the subject has or is at risk of developing a disease or disorder characterized by expression of a RAD54B isoform comprising RAD54B₆₁₈₋₆₂₆. In some embodiments, the disease or condition is cancer. In some embodiments, the cancer is glioma. In some embodiments, the glioma is astrocytoma, oligodendroglioma, or glioblastoma (e.g., glioblastoma multiforme, or GBM). In some embodiments, the glioma is an isocitrate dehydrogenase (IDH)-mutant glioma, such as glioma with mutation in IDH1 or IDH2. In some embodiments, the subject has a mutation in one or more of IDH1, IDH2, MGMT, and EGFR, and/or 1p/19q co-deletion.

In some embodiments, provided herein is a method of inducing an immune response to an isoform of RAD54B comprising the peptide RAD54B₆₁₈₋₆₂₆ (SLYKGLLSV, SEQ ID NO: 1) in a subject, comprising administering to the subject a T-cell comprising or capable of expressing an antigen-binding construct (such as a TCR or antigen-binding derivative or fragment thereof) comprising a) a TCRα variable region comprising i) a CDR1 from the TCRα variable region of SEQ ID NO: 12 or a variant thereof having at least 80% sequence identity to the CDR1 from the TCRα variable region of SEQ ID NO: 12; ii) a CDR2 from the TCRα variable region of SEQ ID NO: 12 or a variant thereof having at least 80% sequence identity to the CDR2 from the TCRα variable region of SEQ ID NO: 12; and/or iii) a CDR3 from the TCRα variable region of SEQ ID NO: 12 or a variant thereof having at least 80% sequence identity to the CDR3 from the TCRα variable region of SEQ ID NO: 12; and/or b) a TCRβ variable region comprising i) a CDR1 from the TCRβ variable region of SEQ ID NO: 14 or a variant thereof having at least 80% sequence identity to the CDR1 from the TCRβ variable region of SEQ ID NO: 14; ii) a CDR2 from the TCRβ variable region of SEQ ID NO: 14 or a variant thereof having at least 80% sequence identity to the CDR2 from the TCRβ variable region of SEQ ID NO: 14; and/or iii) a CDR3 from the TCRβ variable region of SEQ ID NO: 14 or a variant thereof having at least 80% sequence identity to the CDR3 from the TCRβ variable region of SEQ ID NO: 14. In some embodiments, the TCRα variable region comprises a CDR3 having the amino acid sequence of SEQ ID NO: 8 or a variant thereof having at least 80% sequence identity to the amino acid sequence of SEQ ID NO: 8; and/or the TCRβ variable region comprises a CDR3 having the amino acid sequence of SEQ ID NO: 11 or a variant thereof having at least 80% sequence identity to the amino acid sequence of SEQ ID NO: 11. In some embodiments, the antigen-binding construct is specific for the peptide SLYKGLLSV (SEQ ID NO: 1) in a peptide/WIC complex. In some embodiments, the WIC molecule in the peptide/WIC complex is HLA-A*02:01. In some embodiments, the antigen-binding construct is a TCR or antigen-binding derivative or fragment thereof. In some embodiments, the T-cell is human. In some embodiments, the T-cell is a CD8+ T-cell.

In some embodiments, provided herein is a method of inducing an immune response to an isoform of RAD54B comprising the peptide RAD54B₆₁₈₋₆₂₆ (SLYKGLLSV, SEQ ID NO: 1) in a subject, comprising administering to the subject a T-cell comprising or capable of expressing an antigen-binding construct (such as a TCR or antigen-binding derivative or fragment thereof) comprising a TCRα variable region comprising the amino acid sequence of SEQ ID NO: 12 or a variant thereof having at least 80% sequence identity to the amino acid sequence of SEQ ID NO: 12 and/or a TCRβ variable region comprising the amino acid sequence of SEQ ID NO: 14 or a variant thereof having at least 80% sequence identity to the amino acid sequence of SEQ ID NO: 14. In some embodiments, the antigen-binding construct is specific for the peptide SLYKGLLSV (SEQ ID NO: 1) in a peptide/WIC complex. In some embodiments, the WIC molecule in the peptide/MHC complex is HLA-A*02:01. In some embodiments, the antigen-binding construct is a TCR or antigen-binding derivative or fragment thereof. In some embodiments, the T-cell is human. In some embodiments, the T-cell is a CD8+ T-cell.

In some embodiments, according to any of the methods of inducing an immune response described herein, the construct further comprises a TCRα constant region and/or a TCRβ constant region. In some embodiments, the TCRα constant region comprises the amino acid sequence of SEQ ID NO: 13 or a variant thereof having at least 80% sequence identity to the amino acid sequence of SEQ ID NO: 13 and/or the TCRβ constant region comprises the amino acid sequence of SEQ ID NO: 15 or a variant thereof having at least 80% sequence identity to the amino acid sequence of SEQ ID NO: 15. In other embodiments, the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

In some embodiments, according to any of the methods of inducing an immune response described herein, the construct is a multimer comprising i) a first polypeptide comprising the TCRα variable region, and ii) a second polypeptide comprising the TCRβ variable region. In some embodiments, the first polypeptide further comprises any other TCRα sequences present in the antigen-binding construct and/or the second polypeptide further comprises any other TCRβ sequences present in the antigen-binding construct. In some embodiments, the first polypeptide comprises the amino acid sequence of SEQ ID NO: 2 or a variant thereof having at least 80% sequence identity to the amino acid sequence of SEQ ID NO: 2 and/or the second polypeptide comprises the amino acid sequence of SEQ ID NO: 3 or a variant thereof having at least 80% sequence identity to the amino acid sequence of SEQ ID NO: 3. In other embodiments, the first polypeptide comprises the amino acid sequence of SEQ ID NO: 16 or a variant thereof having at least 80% sequence identity to the amino acid sequence of SEQ ID NO: 16 and/or the second polypeptide comprises the amino acid sequence of SEQ ID NO: 17 or a variant thereof having at least 80% sequence identity to the amino acid sequence of SEQ ID NO: 17.

In some embodiments, according to any of the methods of inducing an immune response described herein, the construct is a single chain polypeptide.

In some embodiments, according to any of the methods of inducing an immune response described herein, the immune response is characterized by an increase in IFN-γ levels.

In some embodiments, according to any of the methods of inducing an immune response described herein, the method further comprises administering to the subject RAD54B₆₁₈₋₆₂₆.

In some embodiments, according to any of the methods of inducing an immune response described herein, the subject has or is at risk of developing a disease or condition characterized by expression of a RAD54B isoform comprising RAD54B₆₁₈₋₆₂₆. In some embodiments, the disease or condition is cancer. In some embodiments, the cancer is glioma. In some embodiments, the glioma is astrocytoma, oligodendroglioma, or glioblastoma (e.g., glioblastoma multiforme, or GBM). In some embodiments, the glioma is an isocitrate dehydrogenase (IDH)-mutant glioma, such as glioma with mutation in IDH1 or IDH2. In some embodiments, the subject has a mutation in one or more of IDH1, IDH2, MGMT, and EGFR, and/or 1p/19q co-deletion.

### Methods of Treating a Disease or Condition

In some aspects of the disclosure, an antigen-binding construct (such as a TCR or antigen-binding derivative or fragment thereof) according to any of the embodiments described herein is employed for purposes of treating a disease or condition characterized by an isoform of RAD54B comprising the peptide RAD54B₆₁₈₋₆₂₆ (SLYKGLLSV, SEQ ID NO: 1) in a subject. In some embodiments, the disease or condition is cancer. In some embodiments, the cancer is characterized by expression of a RAD54B isoform comprising RAD54B₆₁₈₋₆₂₆. In some embodiments, the subject has or is at risk of developing a cancer characterized by expression of a RAD54B isoform comprising RAD54B₆₁₈₋₆₂₆. In some embodiments, the cancer is glioma. In some embodiments, the glioma is astrocytoma, oligodendroglioma, or glioblastoma (e.g., glioblastoma multiforme, or GBM). In some embodiments, the glioma is an isocitrate dehydrogenase (IDH)-mutant glioma, such as glioma with mutation in IDH1 or IDH2. In some embodiments, the subject has a mutation in one or more of IDH1, IDH2, MGMT, and EGFR, and/or 1p/19q co-deletion. The antigen-binding construct can be incorporated into a variety of formulations, including those comprising the antigen-binding construct, nucleic acid encoding the antigen-binding construct, and/or T-cells comprising or capable of expressing the antigen-binding construct. More particularly, the antigen-binding construct can be formulated into pharmaceutical compositions by combination with appropriate pharmaceutically acceptable carriers or diluents.

In some embodiments, provided herein is a method of treating a disease or condition characterized by an isoform of RAD54B comprising the peptide RAD54B₆₁₈₋₆₂₆ (SLYKGLLSV, SEQ ID NO: 1) in a subject in need thereof, the method comprising administering to the subject a T-cell comprising or capable of expressing an antigen-binding construct (such as a TCR or antigen-binding derivative or fragment thereof) according to any of the embodiments described herein. In some embodiments, the method further comprises preparing the T-cell, such as by any of the methods of preparing a T-cell described herein. In some embodiments, the T-cells are autologous to the subject. In some embodiments, the T-cells are allogenic to the subject. In some embodiments, the disease or condition is cancer. In some embodiments, the cancer is characterized by expression of a RAD54B isoform comprising RAD54B₆₁₈₋₆₂₆. In some embodiments, the subject has or is at risk of developing a cancer characterized by expression of a RAD54B isoform comprising RAD54B₆₁₈₋₆₂₆. In some embodiments, the cancer is glioma. In some embodiments, the glioma is astrocytoma, oligodendroglioma, or glioblastoma (e.g., glioblastoma multiforme, or GBM). In some embodiments, the glioma is an isocitrate dehydrogenase (IDH)-mutant glioma, such as glioma with mutation in IDH1 or IDH2. In some embodiments, the subject has a mutation in one or more of IDH1, IDH2, MGMT, and EGFR, and/or 1p/19q co-deletion. In some embodiments, the subject is human.

In some embodiments, provided herein is a method of treating a disease or condition characterized by an isoform of RAD54B comprising the peptide RAD54B₆₁₈₋₆₂₆ (SLYKGLLSV, SEQ ID NO: 1) in a subject in need thereof, the method comprising administering to the subject a T-cell comprising or capable of expressing an antigen-binding construct (such as a TCR or antigen-binding derivative or fragment thereof) comprising a) a TCRα variable region comprising i) a CDR1 from the TCRα variable region of SEQ ID NO: 12 or a variant thereof having at least 80% sequence identity to the CDR1 from the TCRα variable region of SEQ ID NO: 12; ii) a CDR2 from the TCRα variable region of SEQ ID NO: 12 or a variant thereof having at least 80% sequence identity to the CDR2 from the TCRα variable region of SEQ ID NO: 12; and/or iii) a CDR3 from the TCRα variable region of SEQ ID NO: 12 or a variant thereof having at least 80% sequence identity to the CDR3 from the TCRα variable region of SEQ ID NO: 12; and/or b) a TCRβ variable region comprising i) a CDR1 from the TCRβ variable region of SEQ ID NO: 14 or a variant thereof having at least 80% sequence identity to the CDR1 from the TCRβ variable region of SEQ ID NO: 14; ii) a CDR2 from the TCRβ variable region of SEQ ID NO: 14 or a variant thereof having at least 80% sequence identity to the CDR2 from the TCRβ variable region of SEQ ID NO: 14; and/or iii) a CDR3 from the TCRβ variable region of SEQ ID NO: 14 or a variant thereof having at least 80% sequence identity to the CDR3 from the TCRβ variable region of SEQ ID NO: 14. In some embodiments, the TCRα variable region comprises a CDR3 having the amino acid sequence of SEQ ID NO: 8 or a variant thereof having at least 80% sequence identity to the amino acid sequence of SEQ ID NO: 8; and/or the TCRβ variable region comprises a CDR3 having the amino acid sequence of SEQ ID NO: 11 or a variant thereof having at least 80% sequence identity to the amino acid sequence of SEQ ID NO: 11. In some embodiments, the antigen-binding construct is specific for the peptide SLYKGLLSV (SEQ ID NO: 1) in a peptide/MHC complex. In some embodiments, the MHC molecule in the peptide/MHC complex is HLA-A*02:01. In some embodiments, the antigen-binding construct is a TCR or antigen-binding derivative or fragment thereof. In some embodiments, the T-cell is human.

In some embodiments, provided herein is a method of treating a disease or condition characterized by an isoform of RAD54B comprising the peptide RAD54B₆₁₈₋₆₂₆ (SLYKGLLSV, SEQ ID NO: 1) in a subject in need thereof, the method comprising administering to the subject a T-cell comprising or capable of expressing an antigen-binding construct (such as a TCR or antigen-binding derivative or fragment thereof) comprising a TCRα variable region comprising the amino acid sequence of SEQ ID NO: 12 or a variant thereof having at least 80% sequence identity to the amino acid sequence of SEQ ID NO: 12 and/or a TCRβ variable region comprising the amino acid sequence of SEQ ID NO: 14 or a variant thereof having at least 80% sequence identity to the amino acid sequence of SEQ ID NO: 14. In some embodiments, the antigen-binding construct is specific for the peptide SLYKGLLSV (SEQ ID NO: 1) in a peptide/MHC complex. In some embodiments, the MHC molecule in the peptide/MHC complex is HLA-A*02:01. In some embodiments, the antigen-binding construct is a TCR or antigen-binding derivative or fragment thereof. In some embodiments, the T-cell is human.

In some embodiments, according to any of the methods of treating a disease or condition described herein, the construct further comprises a TCRα constant region and/or a TCRβ constant region. In some embodiments, the TCRα constant region comprises the amino acid sequence of SEQ ID NO: 13 or a variant thereof having at least 80% sequence identity to the amino acid sequence of SEQ ID NO: 13 and/or the TCRβ constant region comprises the amino acid sequence of SEQ ID NO: 15 or a variant thereof having at least 80% sequence identity to the amino acid sequence of SEQ ID NO: 15. In other embodiments, the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

In some embodiments, according to any of the methods of treating a disease or condition described herein, the construct is a multimer comprising i) a first polypeptide comprising the TCRα variable region, and ii) a second polypeptide comprising the TCRβ variable region. In some embodiments, the first polypeptide further comprises any other TCRα sequences present in the antigen-binding construct and/or the second polypeptide further comprises any other TCRβ sequences present in the antigen-binding construct. In some embodiments, the first polypeptide comprises the amino acid sequence of SEQ ID NO: 2 or a variant thereof having at least 80% sequence identity to the amino acid sequence of SEQ ID NO: 2 and/or the second polypeptide comprises the amino acid sequence of SEQ ID NO: 3 or a variant thereof having at least 80% sequence identity to the amino acid sequence of SEQ ID NO: 3.

In some embodiments, according to any of the host cells comprising or capable of expressing an antigen-binding construct described herein, the construct is a multimer comprising i) a first polypeptide comprising the TCRα variable region, and ii) a second polypeptide comprising the TCRβ variable region. In some embodiments, the first polypeptide further comprises any other TCRα sequences present in the antigen-binding construct and/or the second polypeptide further comprises any other TCRβ sequences present in the antigen-binding construct. In some embodiments, the first polypeptide comprises the amino acid sequence of SEQ ID NO: 16 or a variant thereof having at least 80% sequence identity to the amino acid sequence of SEQ ID NO: 16 and/or the second polypeptide comprises the amino acid sequence of SEQ ID NO: 17 or a variant thereof having at least 80% sequence identity to the amino acid sequence of SEQ ID NO: 17.

In some embodiments, according to any of the methods of treating a disease or condition described herein, the construct is a single chain polypeptide.

In some embodiments, according to any of the methods of treating a disease or condition described herein, the T-cell comprising or capable of expressing an antigen-binding construct is capable of inducing an immune response in the subject when contacted with cells presenting the peptide SLYKGLLSV (SEQ ID NO: 1) in a peptide/WIC complex. In some embodiments, the WIC molecule in the peptide/MHC complex is HLA-A*02:01. In some embodiments, the immune response is characterized by an increase in IFN-γ levels.

In some embodiments, according to any of the methods of treating a disease or condition described herein, the method further comprises administering to the subject RAD54B₆₁₈₋₆₂₆.

The number of administrations of treatment to a subject may vary. In some embodiments, introducing T-cells as provided herein into a subject is a one-time event. In some embodiments, the method further comprises one or more additional administrations of the T-cells.

In some embodiments, provided herein is a method of treating a disease or condition characterized by an isoform of RAD54B comprising the peptide RAD54B₆₁₈₋₆₂₆ (SLYKGLLSV, SEQ ID NO: 1) in a subject in need thereof, the method comprising preparing a T-cell comprising or capable of expressing an antigen-binding construct (such as a TCR or antigen-binding derivative or fragment thereof) according to any of the embodiments described herein, wherein the input T-cell is a cell in the subject.

In some embodiments, according to any of the methods of treating a disease or condition described herein, the disease or condition is cancer. In some embodiments, the cancer is characterized by expression of a RAD54B isoform comprising RAD54B₆₁₈₋₆₂₆. In some embodiments, the subject has or is at risk of developing a cancer characterized by expression of a RAD54B isoform comprising RAD54B₆₁₈₋₆₂₆. In some embodiments, the cancer is glioma. In some embodiments, the glioma is astrocytoma, oligodendroglioma, or glioblastoma (e.g., glioblastoma multiforme, or GBM). In some embodiments, the glioma is an isocitrate dehydrogenase (IDH)-mutant glioma, such as glioma with mutation in IDH1 or IDH2. In some embodiments, the subject has a mutation in one or more of IDH1, IDH2, MGMT, and EGFR, and/or 1p/19q co-deletion.

In some embodiments, provided herein is an antigen-binding construct (such as a TCR or antigen-binding derivative or fragment thereof) according to any of the embodiments described herein, nucleic acid encoding the antigen-binding construct, and/or T-cells comprising or capable of expressing the antigen-binding construct for use in the treatment of a cancer characterized by expression of a RAD54B isoform comprising RAD54B₆₁₈₋₆₂₆, such as for use in the manufacture of a medicament for the treatment of a cancer characterized by expression of a RAD54B isoform comprising RAD54B₆₁₈₋₆₂₆.

### Compositions

In one aspect, some embodiments disclosed herein relate to a composition comprising one or more of: an antigen-binding construct as described herein; nucleic acid encoding an antigen-binding construct as described herein; and a host cell comprising an antigen-binding construct as described herein. In some embodiments, the composition is a pharmaceutical composition and further comprises a pharmaceutically acceptable excipient and/or carrier.

In some embodiments, provided herein are pharmaceutical preparations or compositions comprising an antigen-binding construct (such as a TCR or antigen-binding derivative or fragment thereof) according to any of the embodiments described herein, nucleic acid encoding the antigen-binding construct, and/or T-cells comprising or capable of expressing the antigen-binding construct present in a pharmaceutically acceptable vehicle. “Pharmaceutically acceptable vehicles” may be vehicles approved by a regulatory agency of the Federal or a state government or listed in the US Pharmacopeia or other generally recognized pharmacopeia for use in mammals, such as humans. The term “vehicle” refers to a diluent, adjuvant, excipient, or carrier with which a compound of the disclosure is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the antigen-binding construct, nucleic acid encoding the antigen-binding construct, and/or T-cells comprising or capable of expressing the antigen-binding construct can be achieved in various ways, including oral, buccal, rectal, parenteral, intraperitoneal, intradermal, transdermal, intra-tracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

Pharmaceutical compositions can include, depending on the formulation desired, pharmaceutically acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, non-immunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and/or enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

### Kits

In some embodiments, provided herein are kits for carrying out a method described herein. A kit can include one or more of: an antigen-binding construct as described herein; nucleic acid encoding an antigen-binding construct as described herein; and a host cell comprising an antigen-binding construct as described herein. In some embodiments, the kit further comprises a reagent for reconstituting and/or diluting one or more of the kits components.

A kit as described herein can further include one or more additional reagents, where such additional reagents can be selected from: a dilution buffer; a reconstitution solution; a wash buffer; a control reagent; a control expression vector or polyribonucleotide; a reagent for in vitro production of the antigen-binding construct from DNA, and the like.

Components of a kit can be in separate containers; or can be combined in a single container.

In addition to the above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or sub-packaging) etc. In some embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1. Identification of Candidate GBM Antigen

This examples describes the identification of a target antigen for use in immunotherapy to treat glioblastoma (GBM).

Candidate target antigens for use in the treatment of GBM should have specific and restricted expression in the tumor tissue. Additional features that may be considered for candidate target antigens include having a role in the oncogenic behavior of the tumor and being able to robustly engage the immune system.

Starting from a set of 64 non-mutated peptides from a library of HLA-class I non-mutated peptides derived from primary WHO Grade IV glioma samples (GAPVAC-Peptide Warehouse, Immatics Biotechnologies GmbH), a single-cell RNA sequencing (scRNA-seq) dataset including transcriptomic data from 466 cortical cells clustered into different subpopulations based on their transcriptomic programs (Darmanis, S., et al. (2015). Proceedings of the National Academy of Sciences, 112(23), 7285-7290) was used to narrow the set to 8 candidate peptides. Selection criteria for these candidate peptides included a) expression of the peptide in less than 20% of surveyed cells and b) a level of expression that fell into the three lower quartiles. Next, an scRNA-seq dataset based on sub-cortical cells including gene expression levels at an exon-aligned resolution (Allen Institute of Brain Science) was used to further narrow the set to 4 candidates. Finally, RNA-seq data for expression in peripheral organs from the Genotype-Tissue Expression (GTEx) project was used to narrow the set to 3 candidate peptides. Selection of these candidate peptides was made based on a Median Read Per Count value of less than 1 for the exon encoding the peptide. One of the candidate peptides, a peptide from RAD54B (SLYKGLLSV, SEQ ID NO: 1), a protein involved in DNA repair and recombination, was selected for further analysis.

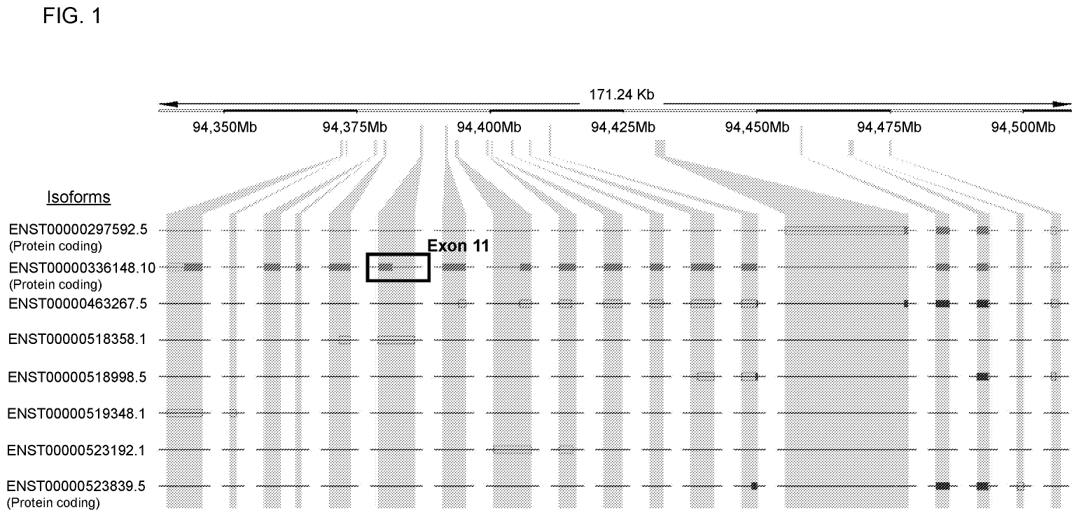

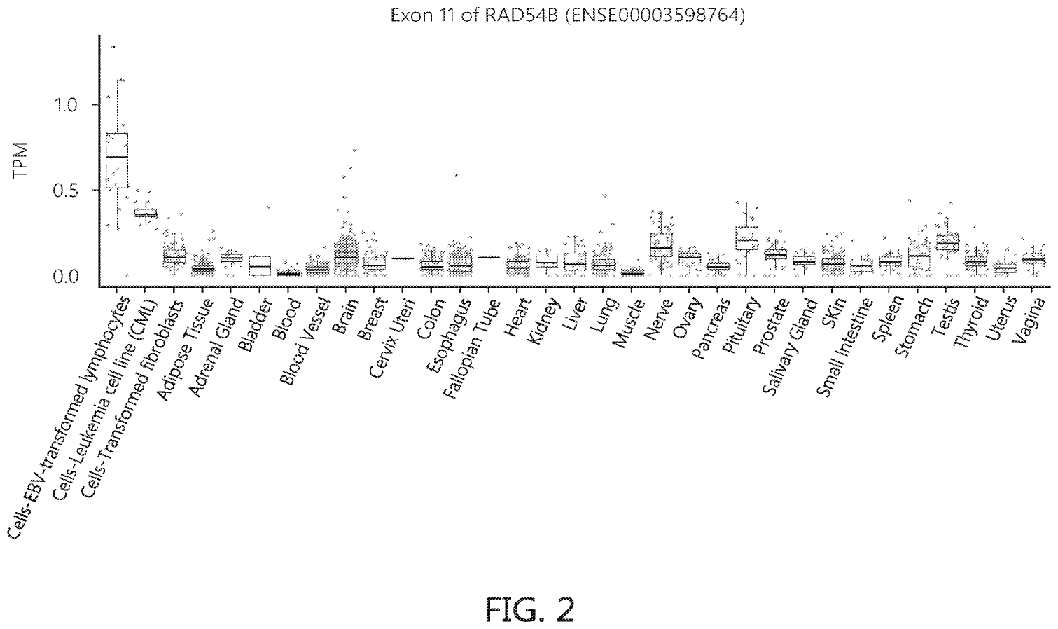

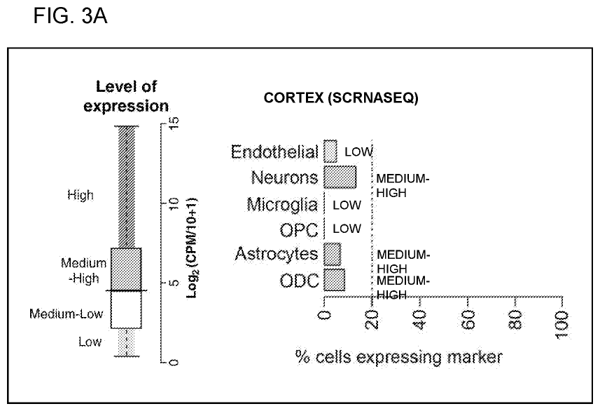

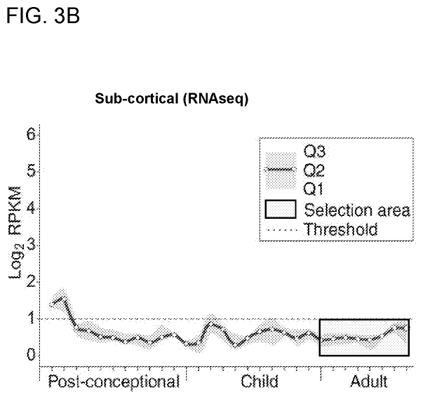

A gene expression model of the RAD54B protein by various isoforms is shown in FIG. 1. Exon 11 in isoform ENST00000336148.10, according to the NCBI or Ensembl database nomenclature, which encodes the peptide of interest (RAD54B₆₁₈₋₆₂₆) is indicated in the box. The candidate RAD54B peptide, referred to herein as RAD54B₆₁₈_626, is encoded on exon 11 of the RAD54B gene. In GTEx nomenclature, the exon is numbered as exon 13. As can be seen from the model shown in FIG. 1, exon 11 is not included in RAD54B expressed in normal tissues, but is present in all three RAD54B isoforms from glioma. FIG. 2 analyzed the RAD54B gene expression in normal peripheral organs using GTEx database. As shown in FIG. 2, the expression of RAD54B is detected in several normal tissues, although at very low levels. Further, it has been shown that the peptide (RAD54B₆₁₈₋₆₂₆) presentation on HLA-A*02:01 was detected only in cancer specimens but not in normal tissues. The data suggests that this epitope can be a target of CAR-T therapy. Additionally, it has been shown that this peptide presentation is up-regulated by irradiation in certain GBM cell lines, for example, U87MG and T98G. Because most of GBM patients undergo radiotherapy after surgical resection, this result supports the usefulness of the therapeutic strategy especially in the clinical setting. The expression profile of RAD54B₆₁₈₋₆₂₆ in the cortex and sub-cortical areas based on the scRNA-seq datasets described above is shown in FIGS. 3A and 3B, respectively. Based on the GTEx database, RAD54B₆₁₈₋₆₂₆ is not expressed in peripheral organs.

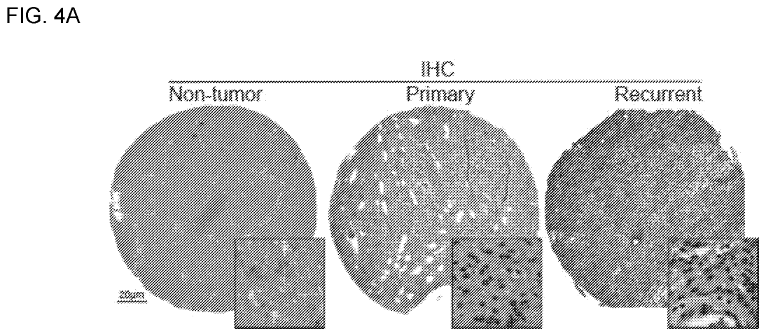

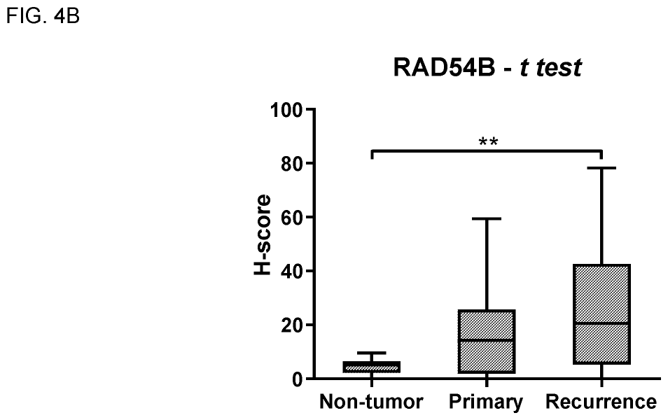

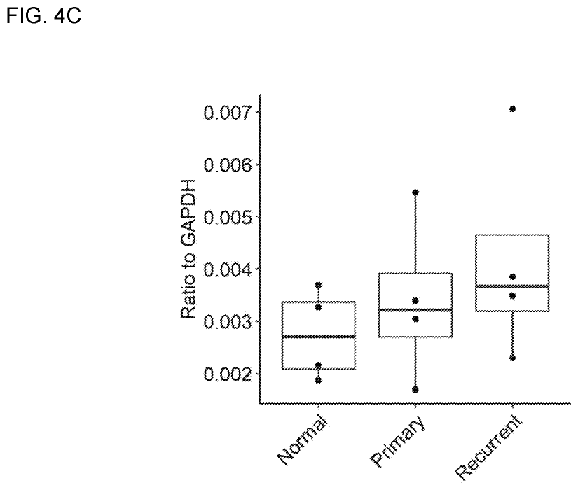

The tumor-specific expression of RAD54B isoforms including exon 11 was validated using immunohistochemistry (IHC) on samples from a cohort of primary and recurrent glioma cases that were paired from the same patients, along with normal cortex samples. Briefly, immunohistochemistry was performed using commercially available antibodies that target the portion of RAD54B including RAD54B₆₁₈₋₆₂₆ following previously described staining methods (Han, S. J., et al. (2016). Journal of neuro-oncology, 130(3), 543-552). As shown in FIGS. 4A-4C, the IHC results show expression of RAD54B₆₁₈₋₆₂₆ in primary and recurrent tumors, but not normal brain, in agreement with the published datasets.

The presentation profile of RAD54B₆₁₈₋₆₂₆ was also evaluated in GBM cells and healthy tissues (adipose, adrenal, bile duct, blood cells, blood vessel, bone marrow, brain, breast, central nerve, esophagus, eye, gallbladder, head & neck, heart, kidney, large intestine, liver, lung, lymph node, ovary, pancreases, parathyroid, peripheral nerve, peritoneum, pituitary, placenta, pleura, prostate, skeletal muscle, skin, small intestine, spinal cord, spleen, stomach, testis, thymus, thyroid, trachea, ureter, urinary bladder, and uterus) by mass spectrometry. RAD54B₆₁₈₋₆₂₆ was found to be presented in tumor cells, but not in any of the healthy tissues, further supporting the tumor-specific expression of this candidate peptide (data not shown).

### Example 2. Generation and Selection of RAD54B-Specific T-Cell Receptor

This examples describes the generation and selection of a T-cell receptor clone recognizing a cancer-specific isoform of RAD54B including the peptide SLYKGLLSV (SEQ ID NO: 1), referred to herein as RAD54B₆₁₈₋₆₂₆.

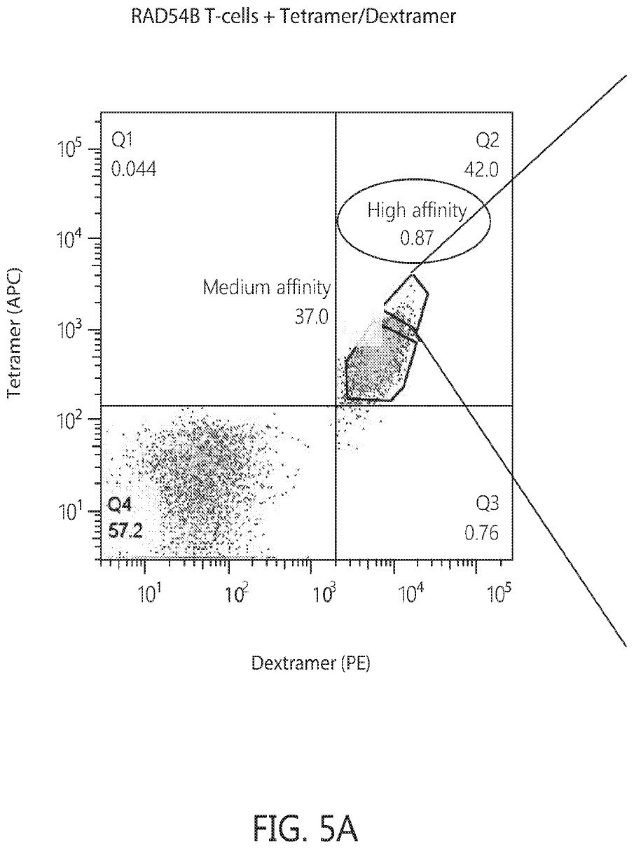

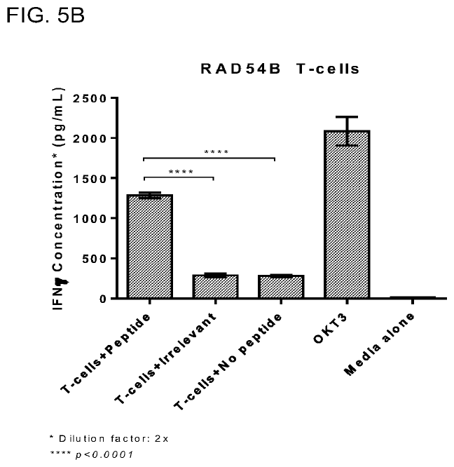

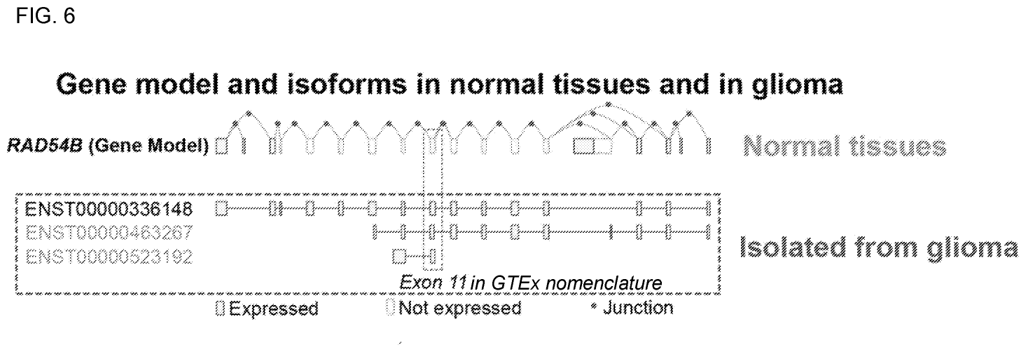

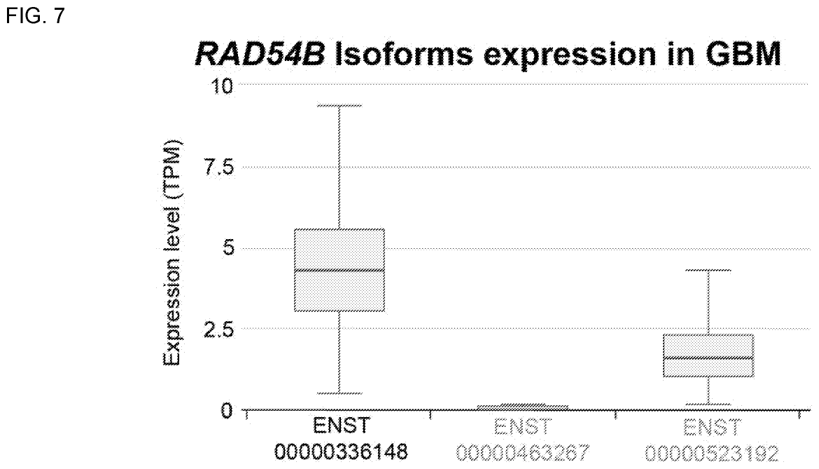

HLA-A*02:01 healthy-donor PBMCs were isolated using density gradient centrifugation methods as previously described (PMID: 19941108). Monocytes at 0.5×10⁶ cell/mL were differentiated and matured into dendritic cells in the presence of 1000 IU IL-4, 1000 IU GM-CSF, 1000 U/mL IFNγ and 250 ng/mL LPS as previously described (Kalinski, P., et al. (2010). Dendritic Cell Protocols (pp. 117-133). Humana Press). Dendritic cells were collected and pulsed with 10 μg/mL of cancer-specific RAD54B peptide RAD54B₆₁₈₋₆₂₆ (SLYKGLLSV, SEQ ID NO: 1) for 1 hour at 37° C. and then co-cultured with 2×10⁶ T-cells from the same donor in the presence of 60 ng/mL IL-21. 5 ng/mL of IL-7 and IL-15 were added every 3 to 4 days and 5×10⁶ cells/mL dendritic cells were added to the culture for peptide re-stimulation every 10 days. T-cell cultures were re-stimulated 4 times using the same concentration of cytokines, cells, and intervals as previously described. After the fourth round of re-stimulation T-cells were collected, stained with anti-CD8 antibody and peptide-specific tetramer and dextramer, and triple-positive cells were isolated by flow cytometry with CD8+ gating followed by selection for high affinity tetramer and dextramer binding (FIG. 5A). Functionality of the isolated T-cell clones was evaluated using ELISpot and IFNγ ELISA. As shown in FIG. 5B, the T-cells were selectively stimulated to secrete IFNγ when incubated with the RAD54B₆₁₈₋₆₂₆ peptide, demonstrating the antigen specificity of the TCRs expressed by these T-cells. The triple-positive cells were sequenced by V(D)J single-cell sequencing using the 10× Genomics platform (10× Genomics). The resulting data were analyzed using the CellRanger pipeline and TCR sequences were identified using the VDJ Loupe by 10× Genomics. One predominant clone, RAD54B₆₁₈₋₆₂₆-specific TCR clone 1, was identified, having a TCRα (TRAV29DV5 TRAJ49 TRAC) sequence of SEQ ID NO: 18 and a TCRβ (TRBV2 TRBD1 TRBJ2-5 TRAC) sequence of SEQ ID NO: 19.

Based on the functionality of T-cells expressing RAD54B₆₁₈₋₆₂₆-specific TCR clone 1 as demonstrated in the ELISpot and IFNγ ELISA assays, this TCR and antigen-binding variants thereof are good candidates for use in therapeutic methods of treating cancers characterized by expression of isoforms of RAD54B containing the RAD54B₆₁₈₋₆₂₆ peptide, such as adoptive T-cell therapy with T-cells engineered to express the TCR or related construct.

### Example 3. Characterization of RAD54B₆₁₈₋₆₂₆-Specific T-Cell Receptors

This example describes methods for the further functional characterization of T-cell receptors and related constructs recognizing RAD54B₆₁₈₋₆₂₆.

### T-Cell Isolation

Healthy donor-derived HLA-A*02:01⁺ PBMCs are obtained from the Stanford Blood Bank (Stanford, Calif.). Patient-derived PBMCs are obtained through the IRB-approved Neurosurgery Tissue Bank (IRB/CHR #10-01318; PI Dr. Joanna Phillips) with coded tissue information without any protected health identifiers. T-cells are enriched from whole blood by immunodensity isolation using the RosetteSep™ Human T-cell Enrichment Cocktail (Stemcell Technologies; 15061) according to the manufacturer's suggested protocol. T-cells are cryopreserved in RPMI media containing 20% human AB serum and 10% DMSO and stored at −196° C.

### ELISPOT Assay

Patient-derived and healthy donor-derived PBMCs are stimulated with 10 μg/ml RAD54B peptide or control irrelevant peptide, flu peptide or without peptide. At 48 hours, rhIL-2 (50 U/ml), IL-7 (10 ng/ml) and IL-15 (10 ng/ml) are added to the culture for an additional 5 days. Fifty thousand peptide stimulated T-cells are co-cultured with 5×10⁴ T2 cells pulsed with 10 μg/ml RAD54B peptide, irrelevant peptide, or without peptide for 24 hrs on the anti-human IFN-γ-antibody-coated ELISPOT plates. To determine TCR avidity, 5×10⁴ TCR-transduced CD8⁺ T-cells are co-cultured with 5×10⁴ T2 cells pulsed with different concentrations of the RAD54B peptide overnight on the anti-human IFN-γ antibody-coated ELISPOT plates. The rest of the protocol is carried out according to the manufacturer's protocol (Human IFN-γ ELISPOT kit, BD, 552138). The spots are quantified using the CTL S6 Universal-V Analyzer ELISpot Reader (ImmunoSpot®).

### HLA-A*0201-Peptide Tetramer Staining

Phycoerythrin (PE)-conjugated HLA-A*0201 RAD54B Dextramer is purchased from Immudex (Denmark). Cells are stained with tetramer (10 μg/mL) or Dextramer in PBS containing 1% bovine serum albumin for 15 minutes at 4° C. (for tetramer) or room temperature (for dextramer), followed by surface staining for various T-cell markers at 4° C. Cells are then blended with PBS containing 0.1% bovine serum albumin. For some experiments, T-cells are stained with tetramer, followed by anti-human CD3 FITC (Biolegend, 344803), CD4-PerCPCy.5.5 (Biolegend, 317427), CD8 APC (Biolegend, 344722), CD69 FITC (eBioscience, 11-0699-42), and/or PD-1-PECy7 (BD-Biosciences, 561272) along with the suitable isotype control antibodies. Intracellular cytokine staining is performed using Fixation/Permeabilization Solution Kit (BD-Biosciences 554714) according to manufacturer's instructions. T-cells are then stained with anti-human Granzyme-B-BV421 (BD-Biosciences, 563389). The cells are acquired using Sony SH800 flow cytometer and analyzed using FlowJo software Version 10.

### ELISA

Media is collected and centrifuged at 500 g for 10 minutes to remove debris. The human IFN-γ (BD OptEIA, 555142) and human IL-2 (Thermo Fischer Scientific, EH2IL2) ELISAs are carried out according to the manufacturer's protocol. Plates are analyzed on a Biotek Synergy2 microplate reader (Biotek) at wavelengths of 450 nm and a background of 550 nm.

### Production of Recombinant TCR

Matured RAD54B TCR α and β ectodomains with an engineered C domain interchain disulfide bond is separately cloned into the pAcGP67a insect expression vector (BD Bio-sciences, 554756) encoding either a C-terminal acidic zipper-Biotin acceptor peptide-6×His tag or a C-terminal basic zipper-6×His tag. A 3c protease site is introduced between the C-terminal TCR (a or β) ectodomain and (acidic or basic) zipper sequence (Birnbaum et al., 2014). Baculoviruses for each TCR construct is produced in SF9 cells. TCR production is carried out in High Five cells by transfecting with appropriate ratio of TCRα and TCRβ viruses for 48-72 hours at 30° C. Harvested culture media is incubated with Ni-NTA resin (QIAGEN 30250) at room temperature for 3 hours and eluted in 1×HBS+200 mM imidazole (pH 7.2). Eluted TCR is buffer-exchanged to 1×HBS and incubate with appropriate amount (100 ng/ul) 3 C protease at 4 degree overnight. The reaction is then purified via size-exclusion chromatography using an AKTAPurifier (GE Healthcare) on a Superdex 200 column (GE Healthcare). Peak fractions are pooled and run SDS-PAGE gel as quality control.

Soluble HLA-A2 loaded with RAD54B peptide is prepared by in vitro folding. The HLA-A2 heavy chain (residues 1-275) and (32 microglobulin (residues 1-100) are separately cloned into pET-26b vector and transformed by Rosseta DE3 E. coli cells. Inclusion bodies containing corresponding proteins are dissolved in 8 M Urea, 50 mM Tris-HCl (pH 8.0), 10 mM EDTA, and 10 mM DTT. For in vitro folding, the HLA-A2 heavy chain, β2-microglobulin and RAD54B peptide are mixed in a 1:2:10 molar ratio and diluted into a folding buffer containing 0.4 M L-arginine-HCl, 100 mM Tris-HCl (pH 8.0), 5 mM EDTA, 0.5 mM Oxidized Glutathione and 5 mM Reduced Glutathione (Garboczi et al., 1992). After 72 hours at 4° C. dialyze against 10 L of 10 mM Tris-HCl, the folding mixture is subjected on a weak ion exchange column (DEAE cellulose). Folded RAD54B-HLA-A2 is purified using sequential size exclusion chromatography (Superdex 200 column) and ion-exchange chromatography (Mono Q columns).

### Surface Plasmon Resonance Analysis

The interaction of the TCR with RAD54B peptide-HLA-A2 is measured by surface plasmon resonance using a BIAcore T100 biosensor at 25° C. Biotinylated RAD54B-HLA-A2 is immobilized on a streptavidin-coated BIAcore SA chip (GE Healthcare) at 300 resonance units (RU). A different flow cell is immobilized with non-relevant peptide-HLA-A2 to serve as blank control. Different concentrations 1H5 TCR solution are flowed sequentially over blank and RAD54B-HLA-A2. Injections of TCR are stopped after 60 s after injections start to allow sufficient time for SPR signals to reach plateau. Dissociation constant (K_{D}) is obtained by fitting equilibrium data with a 1:1 binding model using BIAcore evaluation software.

Infection of Primary T-Cells with TCR Vector

Human PBMCs are activated on plates pre-coated with anti-human CD3 antibody (OKT3 clone, Miltenyi Biotec, 170-076-124) and RetroNectin® (RN, Takara Bio, T100A). Three days after the stimulation, viral supernatant (for TCR-transduction groups) or PBS (for mock-transduction groups) is spun on separate RetroNectin-coated plates at 2,000 g for 2 hrs at 4° C. Activated PBMCs are harvested from the OKT3-Retronectin plates and added to the virus-coated plates using spinfection methodology at 1,000 g for 10 mins at 4° C., and the cells are supplemented with 600 U/ml IL-2 (Peprotech, 200-02). This transduction protocol is repeated on the next day, and PBMCs are allowed to rest for an additional 4 days and then stained with HLA-A*02:01-RAD54B dextramer to determine the transduction efficiency. The T-cells are maintained in 100 U/ml rhIL-2-containing freshly made GT-T551 media (Takara Bio, WK551S).

### LDH-Based Cytotoxicity Assay

The CytoTox 96 non-radioactive cytotoxicity assay (Promega) is carried out according to the manufacturer's protocol. Target cells are plated in 96 well plates with various Effector to Target ratios in 200 μl media for 24 hours. Fifty μl of supernatant is then transferred to an enzymatic assay plate containing 50 μl of CytoTox 96 Reagent and incubated for 30 minutes at room temperature. Stop solution is then added to each well and plates are analyzed at 490 nm on a Synergy2 microplate reader (Biotek). Percent cytotoxicity is calculated as [(Experimental−Effector spontaneous−Target Spontaneous)/(Target Maximum−Target Spontaneous)]×100.

### CSFE-Based Cytotoxicity Assay

Target cells are stained with carboxyfluorescein succinimidyl ester (CF SE) using the Vybrant® CFDA SE Cell Tracer Kit (Thermo Fisher Scientific, V12883). CFSE-labelled target cells (5×10⁴/well) are incubated with CTLs at the E/T ratio of 5 for 8 hours. To block HLA-A2-mediated lysis, anti-HLA-A2 antibody (10 μg/ml, Biolegend, 343302) is added to one group per experiment. At the end of incubation, 7-AAD (Biolegend, 420403) is added into each well and incubated for 10 minutes on ice. The samples are analyzed by flow cytometry, and the killed target cells are identified as CFSE⁺7-AAD⁺ cells. The cytotoxicity is calculated as the percentage of CFSE⁺ and 7-AAD⁺ cells in total CFSE⁺ cells.

### Therapy of Mice Bearing Intracranial Glioma Xenografts

Five- to 6-week-old NOD.Cg-Prkdc^{scid}Il2rg^{tm1Wjl/}SzJ (NSG mice) female mice (Jackson Laboratory, Bar Harbor, Me.) are used in the experiments. Animals are handled in the Animal Facility at the University of California, San Francisco per an Institutional Animal Care and Use Committee-approved protocol. The procedure has been previously described by us (Ohno et al., 2013). Briefly, using a stereotactic apparatus, mice received 5×10⁴ U87 luciferase expressing cells/mouse in 2 μl PBS at 2 mm lateral to the bregma and 3 mm below the surface of the skull. After tumors are established, each mouse received intravenous infusion of PBS, mock-transduced T-cells or 5×10⁶ TCR-transduced via the tail vein on Days 14 and 30-post tumor inoculation. In some experiments, mice are euthanized at 2 days or 10 days post T-cell transfusion. Spleen, blood, and lung tumors are harvested and enumerated for CD8, CD4, Tetramer, Granzyme-B and PD-1.

### Bioluminescence Imaging

The growth of luciferase positive U87 tumors in the brain is non-invasively monitored by BLI using the in vivo imaging system IVIS 100 (PerkinElmer, Alameda, Calif.). Mice receiving intraperitoneal injection with 200 μl(15 mg/ml) of aqueous solution of D-Luciferin potassium salt (PerkinElmer) are anesthetized with isoflurane, and imaged for bioluminescence for 1 min exposure time. The imaging of tumors is carried out in a blinded fashion. Optical images are analyzed by IVIS Living Image software package.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

SEQUENCE LISTING

| SEQ | | |
|---|---|---|
| ID | | |
| NO | Sequence | Description |
| 1 | SLYKGLLSV | RAD54B |
| | | peptide |
| 2 | MAMLLGASVLILWLQPDWVN | TCRα, |
| | SQQKNDDQQVKQNSPSLSVQ | variable and |
| | EGRISILNCDYTNSMFDYFL | constant |
| | WYKKYPAEGPTFLISISSIK | regions, |
| | DKNEDGRFTVFLNKSAKHLS | amino acid |
| | LHIVPSQPGDSAVYFCAASA | |
| | NTGNQFYFGTGTSLTVIPNI | |
| | QNPDPAVYQLRDSKSSDKSV | |
| | CLFTDFDSQTNVSQSKDSDV | |
| | YITDKTVLDKRSMDFKSNSA | |
| | VAWSNKSDFACANAFNNSII | |
| | PEDTFFPSPESSCDVKLVEK | |
| | SFETDTNLNFQ | |
| 16 | KAMLLGASVLILWLQPDWVN | TCRα, |
| | SQQKNDDQQVKQNSPSLSVQ | variable and |
| | EGRISILNCDYTNSMFDYFL | constant |
| | WYKKYPAEGPTFLISISSIK | regions, |
| | DKNEDGRFTVFLNKSAKHLS | full length |
| | LHIVPSQPGDSAVYFCAASA | amino acid |
| | NTGNQFYFGTGTSLTVIPNI | |
| | QNPDPAVYQLRDSKSSDKSV | |
| | CLFTDFDSQTNVSQSKDSDV | |
| | YITDKTVLDMRSMDFKSNSA | |
| | VAWSNKSDFACANAFNNSII | |
| | PEDTFFPSPESSCDVKLVEK | |
| | SFETDTNLNFQNLSVIGFRI | |
| | LLLKVAGFNLLMTLRLWSS | |
| 3 | MDTWLVCWAIFSLLKAGLTE | TCRβ, |
| | PEVTQTPSHQVTQMGQEVIL | variable and |
| | RCVPISNHLYFYWYRQILGQ | constant |
| | KVEFLVSFYNNEISEKSEIF | regions, |
| | DDQFSVERPDGSNFTLKIRS | amino acid |
| | TKLEDSAMYFCASSEGTGGQ | |
| | ETQYFGPGTRLLVLEDLKNV | |
| | FPPEVAVFEPSEAEISHTQK | |
| | ATLVCLATGFYPDHVELSWW | |
| | VNGKEVHSGVSTDPQPLKEQ | |
| | PALNDSRYCLSSRLRVSATF | |
| | WQNPRNHFRCQVQFYGLSEN | |
| | DEWTQDRAK | |
| 17 | MDTWLVCWAIFSLLKAGLTE | TCRβ, |
| | PEVTQTPSHQVTQMGQEVIL | variable and |
| | RCVPISNHLYFYWYRQILGQ | constant |
| | KVEFLVSFYNNEISEKSEIF | regions, |
| | DDQFSVERPDGSNFTLKIRS | full length |
| | TKLEDSAMYFCASSEGTGGQ | amino acid |
| | ETQYFGPGTRLLVLEDLKNV | |
| | FPPEVAVFEPSEAEISHTQK | |
| | ATLVCLATGFYPDHVELSWW | |
| | VNGKEVHSGVSTDPQPLKEQ | |
| | PALNDSRYCLSSRLRVSATF | |
| | WQNPRNHFRCQVQFYGLSEN | |
| | DEWTQDRAKPVTQIVSAEAW | |
| | GRADCGFTSESYQQGVLSAT | |
| | ILYEILLGKATLYAVLVSAL | |
| | VLMAKVKRKDSRG | |
| 4 | ATGGCCATGCTCCTGGGGGC | TCRα, |
| | ATCAGTGCTGATTCTGTGGC | partial |
| | TTCAGCCAGACTGGGTAAAC | nucleotide |
| | AGTCAACAGAAGAATGATGA | |
| | CCAGCAAGTTAAGCAAAATT | |
| | CACCATCCCTGAGCGTCCAG | |
| | GAAGGAAGAATTTCTATTCT | |
| | GAACTGTGACTATACTAACA | |
| | GCATGTTTGATTATTTCCTA | |
| | TGGTACAAAAAATACCCTGC | |
| | TGAAGGTCCTACATTCCTGA | |
| | TATCTATAAGTTCCATTAAG | |
| | GATAAAAATGAAGATGGAAG | |
| | ATTCACTGTCTTCTTAAACA | |
| | AAAGTGCCAAGCACCTCTCT | |
| | CTGCACATTGTGCCCTCCCA | |
| | GCCTGGAGACTCTGCAGTGT | |
| | ACTTCTGTGCAGCAAGCGCG | |
| | AACACCGGTAACCAGTTCTA | |
| | TTTTGGGACAGGGACAAGTT | |
| | TGACGGTCATTCCAAATATC | |
| | CAGAACCCTGACCCTGCCGT | |
| | GTACCAGCTGAGAGACTCTA | |
| | AATCCAGTGACAAGTCTGTC | |
| | TGCCTATTCACCGATTTTGA | |
| | TTCTCAAACAAATGTGTCAC | |
| | AAAGTAAGGATTCTGATGTG | |
| | TATATCACAGACAAAACTGT | |
| | GCTAGACATGAGGTCTATGG | |
| | ACTTCAAGAGCAACAGTGCT | |
| | GTGGCCTGGAGCAACAAATC | |
| | TGACTTTGCATGTGCAAACG | |
| | CCTTCAACAACAGCATTATT | |
| | CCAGAAGACACCTTCTTCCC | |
| | CAGCCCAGAAAGTTCCTGTG | |
| | ATGTCAAGCTGGTCGAGAAA | |
| | AGCTTTGAAACAGATACGAA | |
| | CCTAAACTTTCAAAA | |
| 18 | ATGGCCATGCTCCTGGGGGC | TCRα, full |
| | ATCAGTGCTGATTCTGTGGC | length |
| | TTCAGCCAGACTGGGTAAAC | nucleotide |
| | AGTCAACAGAAGAATGATGA | |
| | CCAGCAAGTTAAGCAAAATT | |
| | CACCATCCCTGAGCGTCCAG | |
| | GAAGGAAGAATTTCTATTCT | |
| | GAACTGTGACTATACTAACA | |
| | GCATGTTTGATTATTTCCTA | |
| | TGGTACAAAAAATACCCTGC | |
| | TGAAGGTCCTACATTCCTGA | |
| | TATCTATAAGTTCCATTAAG | |
| | GATAAAAATGAAGATGGAAG | |
| | ATTCACTGTCTTCTTAAACA | |
| | AAAGTGCCAAGCACCTCTCT | |
| | CTGCACATTGTGCCCTCCCA | |
| | GCCTGGAGACTCTGCAGTGT | |
| | ACTTCTGTGCAGCAAGCGCG | |
| | AACACCGGTAACCAGTTCTA | |
| | TTTTGGGACAGGGACAAGTT | |
| | TGACGGTCATTCCAAATATC | |
| | CAGA | |
| | ACCCTGACCCTGCCGTGTAC | |
| | CAGCTGAGAGACTCTAAATC | |
| | CAGTGACAAGTCTGTCTGCC | |
| | TATTCACCGATTTTGATTCT | |
| | CAAACAAATGTGTCACAAAG | |
| | TAAGGATTCTGATGTGTATA | |
| | TCACAGACAAAACTGTGCTA | |
| | GACATGAGGTCTATGGACTT | |
| | CAAGAGCAACAGTGCTGTGG | |
| | CCTGGAGCAACAAATCTGAC | |
| | TTTGCATGTGCAAACGCCTT | |
| | CAACAACAGCATTATTCCAG | |
| | AAGACACCTTCTTCCCCAGC | |
| | CCAGAAAGTTCCTGTGATGT | |
| | CAAGCTGGTCGAGAAAAGCT | |
| | TTGAAACAGATACGAACCTA | |
| | AACTTTCAAAACCTGTCAGT | |
| | GATTGGGTTCCGAATCCTCC | |
| | TCCTGAAAGTGGCCGGGTTT | |
| | AATCTGCTCATGACGCTGCG | |
| | GCTGTGGTCCAGC | |
| 5 | ATGCCCCTCCTTTCCTCCAC | TCRβ, |
| | AGGACCAGATGCCTGAGCTA | nucleotide |
| | GGAAAGGCCTCATTCCTGCT | (partial, |
| | GTGATCCTGCCATGGATACC | with 5′UTR |
| | TGGCTCGTATGCTGGGCAAT | sequence) |
| | TTTTAGTCTCTTGAAAGCAG | |
| | GACTCACAGAACCTGAAGTC | |
| | ACCCAGACTCCCAGCCATCA | |
| | GGTCACACAGATGGGACAGG | |
| | AAGTGATCTTGCGCTGTGTC | |
| | CCCATCTCTAATCACTTATA | |
| | CTTCTATTGGTACAGACAAA | |
| | TCTTGGGGCAGAAAGTCGAG | |
| | TTTCTGGTTTCCTTTTATAA | |
| | TAATGAAATCTCAGAGAAGT | |
| | CTGAAATATTCGATGATCAA | |
| | TTCTCAGTTGAAAGGCCTGA | |
| | TGGATCAAATTTCACTCTGA | |
| | AGATCCGGTCCACAAAGCTG | |
| | GAGGACTCAGCCATGTACTT | |
| | CTGTGCCAGCAGTGAAGGGA | |
| | CAGGGGGTCAAGAGACCCAG | |
| | TACTTCGGGCCAGGCACGCG | |
| | GCTCCTGGTGCTCGAGGACC | |
| | TGAAAAACGTGTTCCCACCC | |
| | GAGGTCGCTGTGTTTGAGCC | |
| | ATCAGAAGCAGAGATCTCCC | |
| | ACACCCAAAAGGCCACACTG | |
| | GTATGCCTGGCCACAGGCTT | |
| | CTACCCCGACCACGTGGAGC | |
| | TGAGCTGGTGGGTGAATGGG | |
| | AAGGAGGTGCACAGTGGGGT | |
| | CAGCACAGACCCGCAGCCCC | |
| | TCAAGGAGCAGCCCGCCCTC | |
| | AATGACTCCAGATACTGCCT | |
| | GAGCAGCCGCCTGAGGGTCT | |
| | CGGCCACCTTCTGGCAGAAC | |
| | CCCCGCAACCACTTCCGCTG | |
| | TCAAGTCCAGTTCTACGGGC | |
| | TCTCGGAGAATGACGAGTGG | |
| | ACCCAGGATAGGGCCAAACC | |
| 19 | ATGGATACCTGGCTCGTATG | TCRβ, |
| | CTGGGCAATTTTTAGTCTCT | nucleotide |
| | TGAAAGCAGGACTCACAGAA | (full |
| | CCTGAAGTCACCCAGACTCC | length) |
| | CAGCCATCAGGTCACACAGA | |
| | TGGGACAGGAAGTGATCTTG | |
| | CGCTGTGTCCCCATCTCTAA | |
| | TCACTTATACTTCTATTGGT | |
| | ACAGACAAATCTTGGGGCAG | |
| | AAAGTCGAGTTTCTGGTTTC | |
| | CGTTTATAAGAATGAAATCT | |
| | CAGAGAAGTCTGAAATATTC | |
| | GATGATCAATTCTCAGTTGA | |
| | AAGGCCTGATGGATCAAATT | |
| | TCACTCTGAAGATCCGGTCC | |
| | ACAAAGCTGGAGGACTCAGC | |
| | CATGTACTTCTGTGCCAGCA | |
| | GTGAAGGGACAGGGGGTCAA | |
| | GAGACCCAGTACTTCGGGCC | |
| | AGGCACGCGGCTCCTGGTGC | |
| | TCGAGGACCTGAAAAACGTG | |
| | TTCCCACCCGAGGTCGCTGT | |
| | GTTTGAGCCATCAGAAGCAG | |
| | AGATCTCCCACACCCAAAAG | |
| | GCCACACTGGTATGCCTGGC | |
| | CACAGGCTTCTACCCCGACC | |
| | ACGTGGAGCTGAGCTGGTGG | |
| | GTGAATGGGAAGGAGGTGCA | |
| | CAGTGGGGTCAGCACAGACC | |
| | CGCAGCCCCTCAAGGAGCAG | |
| | CCCGCCCTCAATGACTCCAG | |
| | ATACTGCCTGAGCAGCCGCC | |
| | TGAGGGTCTCGGCCACCTTC | |
| | TGGCAGAACCCCCGCAACCA | |
| | CTTCCGCTGTCAAGTCCAGT | |
| | TCTACGGGCTCTCGGAGAAT | |
| | GACGAGTGGACCCAGGATAG | |
| | GGCCAAACCCGTCACCCAGA | |
| | TCGTCAGCGCCGAGGCCTGG | |
| | GGTAGAGCAGACTGTGGCTT | |
| | CACCTCCGAGTCTTACCAGC | |
| | AAGGGGTCCTGTCTGCCACC | |
| | ATCCTCTATGAGATCTTGCT | |
| | AGGGAAGGCCACCTTGTATG | |
| | CCGTGCTGGTCAGTGCCCTC | |
| | GTGCTGATGGCCATGGTCAA | |
| | GAGAAAGGATTCCAGAGGC | |
| 8 | CAASANTGNQFYF | TCRα CDR3 |
| 11 | CASSEGTGGQETQYF | TCRβ CDR3 |
| 12 | MAMLLGASVLILWLQPDWVN | TCRα |
| | SQQKNDDQQVKQNSPSLSVQ | variable |
| | EGRISILNCDYTNSMFDYFL | region |
| | WYKKYPAEGPTFLISISSIK | |
| | DKNEDGRFTVFLNKSAKHLS | |
| | LHIVPSQPGDSAVYFCAASA | |
| | NTGNQFYFGTGTSLTVIPN | |
| 13 | IQNPDPAVYQLRDSKSSDKS | TCRα |
| | VCLFTDFDSQTNVSQSKDSD | constant |
| | VYITDKTVLDMRSMDFKSNS | region |
| | AVAWSNKSDFACANAFNNSI | |
| | IPEDTFFPSPESSCDVKLVE | |
| | KSFETDTNLNFQ | |
| 20 | IQNPDPAVYQLRDSKSSDKS | TCRα |
| | VCLFTDFDSQTNVSQSKDSD | constant |
| | VYITDKTVLDMRSMDFKSNS | region (full |
| | AVAWSNKSDFACANAFNNSI | length) |
| | IPEDTFFPSPESSCDVKLVE | |
| | KSFETDTNLNFQNLSVIGFR | |
| | ILLLKVAGFNLLMTLRLWSS | |
| 14 | KDTWLVCWAIFSLLKAGLTE | TCRβ |
| | PEVTQTPSHQVTQMGQEVIL | variable |
| | RCVPISNHLYFYWYRQILGQ | region |
| | KVEFLVSFYNNEISEKSEIF | |
| | DDQFSVERPDGSNFTLKIRS | |
| | TKLEDSAMYFCASSEGTGGQ | |
| | ETQYFGPGTRLLVLE | |
| 15 | DLKNVFPPEVAVFEPSEAEI | TCRβ |
| | SHTQKATLVCLATGFYPDHV | constant |
| | ELSWWVNGKEVHSGVSTDPQ | region |
| | PLKEQPALNDSRYCLSSRLR | |
| | VSATFWQNPRNHFRCQVQFY | |
| | GLSENDEWTQDRAK | |
| 21 | DLKNVFPPEVAVFEPSEAEI | TCRβ |
| | SHTQKATLVCLATGFYPDHV | constant |
| | ELSWWVNGKEVHSGVSTDPQ | region (full |
| | PLKEQPALNDSRYCLSSRLR | length) |
| | VSATFWQNPRNHFRCQVQFY | |
| | GLSENDEWTQDRAKPVTQIV | |
| | SAEAWGRADCGFTSESYQQG | |
| | VLSATILYEILLGKATLYAV | |
| | LVSALVLMAMVKRKDSRG | |

What is claimed is:

1. An antigen-binding construct comprising
i) a TCRα variable region comprising a complementary determining region (CDR) 3 having the amino acid sequence of SEQ ID NO: 8 or a variant thereof having at least 80% sequence identity to the amino acid sequence of SEQ ID NO: 8; and
ii) a TCRβ variable region comprising a CDR3 having the amino acid sequence of SEQ ID NO: 11 or a variant thereof having at least 80% sequence identity to the amino acid sequence of SEQ ID NO: 11,
wherein the antigen-binding construct is specific for the peptide SLYKGLLSV (SEQ ID NO: 1) in a peptide/MHC complex.

2. The antigen-binding construct of claim 1, wherein
iii) the TCRα variable region further comprises a CDR1 from the TCRα variable region of SEQ ID NO: 12 or a variant thereof having at least 80% sequence identity to the CDR1 from the TCRα variable region of SEQ ID NO: 12;
iv) the TCRα variable region further comprises a CDR2 from the TCRα variable region of SEQ ID NO: 12 or a variant thereof having at least 80% sequence identity to the CDR2 from the TCRα variable region of SEQ ID NO: 12;
v) the TCRβ variable region further comprises a CDR1 from the TCRβ variable region of SEQ ID NO: 14 or a variant thereof having at least 80% sequence identity to the CDR1 from the TCRβ variable region of SEQ ID NO: 14; and/or
vi) the TCRβ variable region further comprises a CDR2 from the TCRβ variable region of SEQ ID NO: 14 or a variant thereof having at least 80% sequence identity to the CDR2 from the TCRβ variable region of SEQ ID NO: 14.

3. The antigen-binding construct of claim 2, wherein the TCRα variable region comprises the amino acid sequence of SEQ ID NO: 12 or a variant thereof having at least 80% sequence identity to the amino acid sequence of SEQ ID NO: 12 and/or the TCRβ variable region comprises the amino acid sequence of SEQ ID NO: 14 or a variant thereof having at least 80% sequence identity to the amino acid sequence of SEQ ID NO: 14.

4. The antigen-binding construct of any one of claims 1-3, wherein the construct further comprises a TCRα constant region comprising the amino acid sequence of SEQ ID NO: 13 or a variant thereof having at least 80% sequence identity to the amino acid sequence of SEQ ID NO: 13 and/or a TCRβ constant region comprising the amino acid sequence of SEQ ID NO: 15 or a variant thereof having at least 80% sequence identity to the amino acid sequence of SEQ ID NO: 15.

5. The antigen-binding construct of claim 4, wherein the TCRα constant region comprises the amino acid sequence of SEQ ID NO: 20 or a variant thereof having at least 80% sequence identity to the amino acid sequence of SEQ ID NO: 20 and/or the TCRβ constant region comprises the amino acid sequence of SEQ ID NO: 21 or a variant thereof having at least 80% sequence identity to the amino acid sequence of SEQ ID NO: 21.

6. The antigen-binding construct of any one of claims 1-5, wherein the construct is a multimer comprising i) a first polypeptide comprising the TCRα variable region comprising comprises the amino acid sequence of SEQ ID NO: 2 or a variant thereof having at least 80% sequence identity to the amino acid sequence of SEQ ID NO: 2, and ii) a second polypeptide comprising the TCRβ variable region comprising the amino acid sequence of SEQ ID NO: 3 or a variant thereof having at least 80% sequence identity to the amino acid sequence of SEQ ID NO: 3.

7. The antigen-binding construct of claim 6, wherein the first polypeptide comprises the amino acid sequence of SEQ ID NO: 16 or a variant thereof having at least 80% sequence identity to the amino acid sequence of SEQ ID NO: 16 and/or the second polypeptide comprises the amino acid sequence of SEQ ID NO: 17 or a variant thereof having at least 80% sequence identity to the amino acid sequence of SEQ ID NO: 17.

8. The antigen-binding construct of any one of claims 1-7, wherein the construct is a T-cell receptor or an antigen-binding derivative or fragment thereof.

9. The antigen-binding construct of any one of claims 1-8, wherein the MHC molecule in the peptide/MHC complex is HLA-A*02:01.

10. A nucleic acid encoding the antigen-binding construct of any one of claims 1-9.

11. A host cell comprising the nucleic acid of claim 10, wherein the antigen-binding construct is capable of being expressed in the host cell.

12. The host cell of claim 11, wherein the nucleic acid encoding the antigen-binding construct is heterologous to the host cell.

13. The host cell of claim 12, wherein the host cell is a T-cell.

14. The host cell of claim 13, wherein the T-cell is a CD8+ T-cell.

15. A method of preparing a T-cell comprising or capable of expressing the antigen-binding-construct of any one of claims 1-9, comprising introducing nucleic acid encoding the antigen-binding construct into an input T-cell, wherein the antigen-binding construct is capable of being expressed in the input T-cell following introduction of the nucleic acid.

16. A method of inducing an immune response to an isoform of RAD54B comprising the peptide SLYKGLLSV (SEQ ID NO: 1) in a subject, comprising administering to the subject a T-cell comprising or capable of expressing the antigen-binding construct of any one of claims 1-9.

17. A method of treating a disease or condition characterized by an isoform of RAD54B comprising the peptide SLYKGLLSV (SEQ ID NO: 1) in a subject in need thereof, comprising administering to the subject a T-cell comprising or capable of expressing the antigen-binding construct of any one of claims 1-9.

18. The method of claim 16 or 17, wherein the T-cell is autologous to the subject.

19. The method of any one of claims 16-18, wherein the subject has or is at risk of developing a cancer characterized by expression of a RAD54B isoform comprising RAD54B₆₁₈₋₆₂₆.

20. The method of claim 19, wherein the cancer is a glioma.

21. The method of claim 20, where the glioma is an astrocytoma, an oligodendroglioma, or a glioblastoma.


SEQUENCE LISTING (21 sequences)

SEQ ID NO: 1 - protein, 9 aa, Homo sapiens
SLYKGLLSV

SEQ ID NO: 2 - protein, 251 aa, Artificial Sequence
  Synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASANTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQ

SEQ ID NO: 3 - protein, 249 aa, Artificial Sequence
  Synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEGTGGQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAK

SEQ ID NO: 4 - DNA, 755 bp, Artificial Sequence
  Synthetic construct
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgcgaacaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaa

SEQ ID NO: 5 - DNA, 820 bp, Artificial Sequence
  Synthetic construct
atgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagggacagggggtcaagagacccagtacttcgggccaggcacgcggctcctggtgctcgaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacc

SEQ ID NO: 6-7 - skipped (2 entries)

SEQ ID NO: 8 - protein, 13 aa, Artificial Sequence
  Synthetic construct
CAASANTGNQFYF

SEQ ID NO: 9-10 - skipped (2 entries)

SEQ ID NO: 11 - protein, 15 aa, Artificial Sequence
  Synthetic construct
CASSEGTGGQETQYF

SEQ ID NO: 12 - protein, 139 aa, Artificial Sequence
  Synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASANTGNQFYFGTGTSLTVIPN

SEQ ID NO: 13 - protein, 112 aa, Artificial Sequence
  Synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQ

SEQ ID NO: 14 - protein, 135 aa, Artificial Sequence
  Synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEGTGGQETQYFGPGTRLLVLE

SEQ ID NO: 15 - protein, 114 aa, Artificial Sequence
  Synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAK

SEQ ID NO: 16 - protein, 279 aa, Artificial Sequence
  Synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASANTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 313 aa, Artificial Sequence
  Synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEGTGGQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 18 - DNA, 837 bp, Artificial Sequence
  Synthetic construct
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgcgaacaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 19 - DNA, 939 bp, Artificial Sequence
  Synthetic construct
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagggacagggggtcaagagacccagtacttcgggccaggcacgcggctcctggtgctcgaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 20 - protein, 140 aa, Artificial Sequence
  Synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - protein, 178 aa, Artificial Sequence
  Synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG